(19) United States
(12) Patent Application Publication
(10) Pub. No.: US 2025/0297308 A1
(43) Pub. Date: Sep. 25, 2025

(54) METHODS, COMPOSITIONS, AND KITS FOR REDUCING ANALYTE MISLOCALIZATION

(71) Applicant: 10x Genomics, Inc., Pleasanton, CA (US)

(72) Inventor: Augusto Manuel Tentori, Dublin, CA (US)

(21) Appl. No.: 19/230,378

(22) Filed: Jun. 6, 2025

Related U.S. Application Data
(63) Continuation of application No. PCT/US2024/021929, filed on Mar. 28, 2024.
(60) Provisional application No. 63/455,113, filed on Mar. 28, 2023.

(51) Int. Cl. C12Q 1/6874; C12Q 1/6832

(52) U.S. Cl. CPC C12Q 1/6874; C12Q 1/6832

(57) ABSTRACT

Compositions, kits, and methods for reducing mislocalization of analytes from a biological sample in the context of an array-based spatial analysis platform are disclosed herein. Also, disclosed herein are a first region of capture probes, where the capture probes include: (i) a spatial barcode, (ii) a first capture domain, and (iii) one or more functional domains, and a second region of capture probes, where the capture probes include a second capture domain. The second region of capture probes can capture analytes from portions of the biological sample that exceed the boundaries of the first region of capture probes, thereby reducing analyte mislocalization and improving the accuracy of the array-based spatial analysis platform.

### CROSS-REFERENCE TO RELATED APPLICATIONS

Pursuant to 35 U.S.C. § 119(e), this application is a continuation of International Application PCT/US2024/021929, with an international filing date of Mar. 28, 2024, which claims the benefit of priority to U.S. Provisional Patent Application No. 63/455,113, filed on Mar. 28, 2023, the contents of which are hereby incorporated by reference.

### BACKGROUND

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, and signaling and cross-talk with other cells in the tissue.

Spatial heterogeneity has been previously studied using techniques that only provide data for a small handful of analytes in the context of an intact tissue or a portion of a tissue, or provides substantial analyte data for dissociated tissue (i.e., single cells), but fail to provide information regarding the position of the single cell in a parent biological sample (e.g., tissue sample).

High-throughput methods are available for spatial analysis to determine the identity, abundance, and distribution of analytes within cells within a biological sample, for example, a tissue sample or section. Such methods include array-based spatial transcriptomics assays. The biological sample can be placed on a substrate or aligned with an array to improve specificity and efficiency when being analyzed for identification or characterization of an analyte, such as DNA, RNA or other genetic material, within the sample. However, mislocalization of the analyte during the spatial analysis assay can distort the detection signal and reduce the accuracy of the spatial analysis. For example, during spatial analysis, the edges or boundaries of the array can capture analytes or proxies thereof from tissue that exceeded the boundaries of the probes on the array, leading to mislocalization of analytes thereby generating a “hot edge” of captured analytes resulting in an artificially elevated detection signal at the perimeter of the array. Therefore, there exists a need for compositions and methods for reducing mislocalization of analytes in spatial analysis assays.

### SUMMARY

Spatial transcriptomics assays performed using capture probes in an array format, which can include aligning a biological sample with the array resulting in a confined reaction space, can show a “hot edge” signal pattern. In this pattern, particularly when the biological sample extends beyond the boundaries of the capture probes on the array, spots at the edge of the array can have an artificially elevated signal relative to adjacent spots situated in the interior of the array. This elevated signal represents an artifact deviating from the true signal. While not wishing to be bound by theory, it is thought that during transfer of analytes from the biological sample and capture of the analytes by the capture probes on an array, analytes migrating from cells that are aligned with capture probes are captured. Analytes migrating from cells that are not aligned with capture probes are free to diffuse and mislocalize and can be captured by any adjacent capture probe, resulting in the hot edge signal pattern on at least the outermost capture probes at the periphery of the array. For example, this hot edge signal pattern of mislocalized target analytes can occur when the biological sample extends beyond the boundaries of the capture probes on the array.

Disclosed herein are compositions, kits, and methods for reducing and/or preventing the hot edge signal pattern by reducing mislocalization of analytes in spatial analysis assays. The compositions, kits, and methods disclosed herein include capture probes at the periphery of the array that capture analytes but are not ultimately detected, thereby reducing the effect of mislocalization of analytes on spatially derived data and mitigating the hot edge signal pattern in spatial analysis assays. In cases where the biological sample extends beyond the boundaries of the capture probes on the array, including a perimeter of capture probes in the array design and fabrication that capture analytes that are not ultimately detected prevents the artificial elevation that would occur if detecting signal at those spots. As a result, the compositions, kits, and methods disclosed herein improve the accuracy of spatial transcriptomics assays performed using capture probes in an array format.

In a first aspect, the disclosure provides methods for reducing mislocalization of target nucleic acids from a biological sample, the method including: (a) providing an array, wherein the array includes a first region of capture probes and a second region of capture probes, wherein capture probes of the first region of capture probes include: (i) a spatial barcode, (ii) a first capture domain, and (iii) one or more functional domains comprising a primer binding site or a sequencing specific site, and capture probes of the second region of capture probes include a second capture domain; and (b) hybridizing target nucleic acids from the biological sample to the first capture domain of capture probes of the first region and a second capture domain of the capture probes of the second region, wherein the capture probes of the second region do not comprise one or more of a primer binding site or sequencing specific site (i.e., the capture probes in the second region do not include the primer binding site or sequencing specific site as the capture probes in the first region), such that hybridization of the target nucleic acids to the capture probes of the second region of capture probes reduces mislocalization of target nucleic acids.

In some embodiments, the methods further include migrating the target nucleic acids from the biological sample to the array. In some embodiments, the biological sample is disposed on the array. In some embodiments, the biological sample is disposed on a first substrate. In some embodiments, the methods further include aligning the first substrate including the biological sample with a second substrate including the array, such that at least a portion of the biological sample is aligned with at least a portion of the first region on the array.

In some embodiments, capture probes of the first region further include a unique molecular identifier and/or a cleavage domain. In some embodiments, the second region of capture probes surrounds the first region of capture probes, optionally wherein the capture probes of the first region are affixed to features in an interior of the array and capture probes of the second region are affixed to features on the perimeter of the array. In some embodiments, the perimeter of the array includes a peripheral edge of a plurality of patterned features on the spatial array.

In some embodiments, the capture probes of the first region and the capture probes of the second region are affixed to features on the array. In some embodiments, the first capture domain is the same as the second capture domain, optionally wherein the first capture domain and the second capture domain independently include a poly(T) sequence. In some embodiments, the first capture domain and the second capture domain include the same non-homopolymeric sequence.

In some embodiments, the target nucleic acids include mRNA. In some embodiments, the method includes extending the capture probe using the target nucleic acids as a template, thereby generating an extension product (i.e., an extended capture probe). In some embodiments, the method includes generating complementary strands to the extension products.

In some embodiments, the target nucleic acids are ligation products that represents mRNA target nucleic acids. In some embodiments, the ligation products are generated by ligating first probes that hybridize to the target nucleic acids and second probes that hybridize adjacently to the first probes on the target nucleic acids. In some embodiments, either the first probes or the second probes include a capture domain that is complementary to the first capture domain and/or the second capture domain, and either the first probes or the second probes include one or more of a primer binding site or a sequencing specific site. In some embodiments, the ligating is performed by a ligase selected from a PBCV-1 ligase, a Chlorella DNA ligase, a single stranded DNA ligase, or a T4 DNA ligase. In some embodiments, the ligase is T4 DNA ligase or a Chlorella DNA ligase. In some embodiments, the method further comprises releasing the ligation products from the mRNA target nucleic acids, optionally, where the releasing comprises treatment with an RNase. In some embodiments, the method includes extending the ligation products using the capture probes of the first region as a template, thereby generating extension products and/or extending the ligation products using the capture probes of the first region as a template, thereby generating extended ligation products.

In some embodiments, the method includes generating a nucleic acid library for sequencing including either: (i) extension products generated from target nucleic acids, or complements thereof, or (ii) extension products generated from ligation products, or complements thereof.

In some embodiments, the methods further include determining (i) the sequence of the spatial barcode or a complement thereof, and (ii) all or a portion of the sequence of the target nucleic acids, or a complement thereof, or all or a portion of the sequence of the ligation products, or a complement thereof, and using the determined sequences of (i) and (ii) to determine the locations of the target nucleic acids in the biological sample. In some embodiments, the determining includes sequencing. In some embodiments, the sequencing includes high-throughput sequencing. In some embodiments, the method includes sequencing the nucleic acid library with a sequencing primer that hybridizes to the sequencing specific site of capture probes in the first region.

In some embodiments, the methods further include permeabilizing the biological sample. In some embodiments, the permeabilizing includes the use of a protease. In some embodiments, the protease includes pepsin. In some embodiments, the protease includes proteinase K.

In another aspect, the disclosure provides methods of reducing sequencing output from mislocalized target nucleic acids from a biological sample, the methods including: (a) providing an array, wherein the array includes a first region of capture probes and a second region of capture probes, wherein capture probes of the first region include: (i) a spatial barcode, (ii) a first capture domain, and (iii) one or more functional domains, and capture probes of the second region include a second capture domain; (b) contacting target nucleic acids from the biological sample with the plurality of capture probes on the array wherein the capture probes of the first and second regions hybridize to the target nucleic acids in the first and second regions proximal to their location in the biological sample; (c) extending the capture probes hybridized to the target nucleic acids, thereby generating extension products, and (d) determining the sequence of the (i) spatial barcode or a complement thereof and (ii) a portion of the target nucleic acid or a complement thereof from the extension products from capture probes in the first region, thereby reducing sequencing output for mislocalized target nucleic acids from the biological sample.

In some embodiments, the extension products, or complements thereof, from capture probes of the second region are not sequenced, and optionally the second region of capture probes is located around a perimeter of the array and surrounds the first region of capture probes. In some embodiments, the perimeter of the array includes a peripheral edge of a plurality of patterned features on the spatial array.

In some embodiments, the biological sample is disposed on the array. In some embodiments, one or more portions of the biological sample is located over the second region of the array. In some embodiments, the methods further include aligning a first substrate including the biological sample with a second substrate including the array, such that at least a portion of the biological sample is aligned with at least a portion of the first region of the array. In some embodiments, the methods further include migrating the target nucleic acids from the biological sample to the array.

In some embodiments, the one or more functional domains is a primer binding site or a sequencing specific site. In some embodiments, capture probes of the second region do not have a primer binding site or a sequencing specific site. In some embodiments, capture probes of the first region further include one or more of a unique molecular identifier (UMI) and a cleavage domain. In some embodiments, the capture probes of the first region and the capture probes of the second region are affixed to features on the array. In some embodiments, the capture probes of the first region are affixed to features in an interior of the array and capture probes of the second region are affixed to features on the perimeter of the array. In some embodiments, the first capture domain and the second capture domain are the same, optionally where the first capture domain and the second capture domain independently include a poly(T) sequence. In some embodiments, the first capture domain and the second capture domain include the same non-homopolymeric sequence.

In some embodiments, the target nucleic acids comprise mRNA. In some embodiments, the method includes extending the target nucleic acids using the capture probes of the first region as a template, thereby generating extended target nucleic acids.

In some embodiments, the target nucleic acids are ligation products that represents mRNA target nucleic acids. In some embodiments, the ligation products are generated by ligating a first probe that hybridizes to the target nucleic acid sequence and a second probe that hybridizes adjacently to the first probe on the target nucleic acid sequence. In some embodiments, either the first probe or second probe includes a capture probe binding domain that is complementary to the first capture domain and/or the second capture domain of the capture probes on the array. In some embodiments, either the first probe or second probe further includes one or more of a primer binding site or a sequencing specific site. In some embodiments, the ligating is performed by a ligase selected from a PBCV-1 ligase, a Chlorella DNA ligase, a single stranded DNA ligase, or a T4 DNA ligase. In some embodiments, the ligase is T4 DNA ligase or a Chlorella DNA ligase. In some embodiments, the method includes releasing the ligation products from the mRNA target nucleic acids, optionally, wherein the releasing comprises treatment with an RNase.

In some embodiments, the method includes extending the capture probes of the first region using the ligation products as templates, thereby generating extension products and/or extending the ligation products using the capture probes of the first region as templates, thereby generating extended ligation products.

In some embodiments, the method includes generating a nucleic acid library for sequencing including either: (i) extension products generated from target nucleic acids, or complements thereof, or (ii) extension products generated from ligation products, or complements thereof.

In some embodiment, the determining includes sequencing. In some embodiments, the sequencing includes high-throughput sequencing. In some embodiments, the method includes sequencing the nucleic acid library with a sequencing primer that hybridizes to the sequencing specific site of capture probes in the first region.

In some embodiments, the biological sample is a tissue sample. In some embodiments, the tissue sample is a tissue section. In some embodiments, the tissue sample is a fixed tissue sample. In some embodiments, the fixed tissue sample is methanol-fixed, acetone-fixed, PFA-fixed, or is formalin-fixed paraffin-embedded (FFPE). In some embodiments, the FFPE tissue is deparaffinized and decrosslinked prior to step (b). In some embodiments, the tissue sample is a fresh frozen tissue sample.

In some embodiments, the tissue sample is fixed and stained prior to step (b), optionally using immunofluorescence, immunohistochemistry, or hematoxylin and eosin.

In some embodiments, the methods further include permeabilizing the biological sample. In some embodiments, the permeabilizing includes the use of a protease. In some embodiments, the protease includes pepsin. In some embodiments, the protease includes proteinase K.

In another aspect, the disclosure provides a spatial array including: (a) a first plurality of capture probes, wherein each of the first plurality of capture probes includes (i) a spatial barcode, (ii) a first capture domain, and (iii) one or more functional domains; (b) a second plurality of capture probes, wherein each of the second plurality of capture probes includes a second capture domain, and wherein the second plurality of capture probes are positionally located around a perimeter of the array.

In some embodiments, the perimeter includes a peripheral edge of a plurality of patterned features on the spatial array. In some embodiments, the spatial array further includes: (c) a biological sample including a plurality of target nucleic acids on the array; and (d) a first probe and a second probe hybridized to the target nucleic acid and ligated together, wherein the first probe and the second probe each include a sequence that is substantially complementary to adjacent sequences of the target nucleic acid, wherein the first probe and/or the second probe contain one or more functional sequences, and wherein one of the first probe or the second probe includes a capture probe capture domain. In some embodiments, each of the first plurality of capture probes further includes a unique molecular identified (UMI). In some embodiments, the second plurality of capture probes does not include a spatial barcode or a functional domain (e.g., a sequencing specific site or a primer binding site). In some embodiments, each of the first plurality of capture probes and each of the second plurality of capture probes includes a poly(T) sequence (e.g., a poly(T) capture domain sequence). In some embodiments, each of the first plurality of capture probes includes one or more functional domains, a cleavage domain, or a combination thereof. In some embodiments, the first plurality of capture probes and the second plurality of capture probes are affixed to the plurality of patterned features on the array.

In another aspect, the disclosure provides a kit including: (a) a spatial array including a first plurality of capture probes, wherein each of the first plurality of capture probes includes a (i) a spatial barcode, (ii) a first capture domain, and (iii) one or more functional domains; and (b) a second plurality of capture probes, wherein each of the second plurality of capture probes includes a second capture domain; wherein the second plurality of capture probes are configured to surround the first plurality of capture probes on the array. In some embodiments, each of the second plurality of capture probes does not include a spatial barcode or a functional domain (e.g., a sequencing specific site or a primer binding site).

In some embodiments, the kit further includes a polymerase. In some embodiments, the polymerase is one or more of a reverse transcriptase, a DNA polymerase and a ligase. In some embodiments, each of the first plurality of capture probes further includes a unique molecular identifier (UMI), a cleavage domain, or combinations thereof. In some embodiments, the kit further includes one or more permeabilization reagents. In some embodiments, the one more permeabilization reagents includes pepsin or proteinase K. In some embodiments, the kit further includes a RNase. In some embodiments, the RNase is RNase H. In some embodiments, the kit further includes instructions for performing any of the methods provided herein.

Also provided herein are methods for reducing mislocalization of target nucleic acids from a biological sample, the method including: (a) providing an array, where the array includes a first region of capture probes and a second region of capture probes, where capture probes of the first region of capture probes include: (i) a spatial barcode, (ii) a capture domain, and (iii) one or more functional domains, and capture probes of the second region of capture probes comprise a capture domain; and (b) hybridizing target nucleic acids from the biological sample to the capture domains of capture probes of the first region and the capture probes of the second region, such that hybridization of the target nucleic acids to the capture probes of the second region of capture probes reduces mislocalization of target nucleic acids.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “about” or “approximately” as used herein means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to +20%, preferably up to +10%, more preferably up to +5%, and more preferably still up to +1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

The term “substantially complementary” used herein means that a first sequence is at least 50%, 55%, 60%, 65%, 70%, 75%, 80%, 85%, 90%, 95%, 97%, 98% or 99% identical to the complement of a second sequence over a region of 2, 3, 4, 5, 6, 7, 8, 9, 10, 11, 12, 13, 14, 15, 16, 17, 18, 19, 20-40, 40-60, 60-100, or more nucleotides, or that the two sequences hybridize under stringent hybridization conditions. Substantially complementary also means that a sequence in one strand is not completely and/or perfectly complementary to a sequence in an opposing strand, but that sufficient bonding occurs between bases on the two strands to form a stable hybrid complex in set of hybridization conditions (e.g., salt concentration and temperature). Such conditions can be predicted by using the sequences and standard mathematical calculations known to those skilled in the art.

The term “each,” when used in reference to a collection of items, is intended to identify an individual item in the collection but does not necessarily refer to every item in the collection, unless expressly stated otherwise, or unless the context of the usage clearly indicates otherwise.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1A shows an exemplary sandwiching process where a first substrate (e.g., a slide), including a biological sample, and a second substrate (e.g., array slide) are brought into proximity with one another.

FIG. 1B shows a fully formed sandwich configuration creating a chamber formed from the one or more spacers, the first substrate, and the second substrate.

FIG. 2A shows a perspective view of an exemplary sample handling apparatus in a closed position.

FIG. 2B shows a perspective view of an exemplary sample handling apparatus in an open position.

FIG. 3A shows the first substrate angled over (superior to) the second substrate.

FIG. 3B shows that as the first substrate lowers, and/or as the second substrate rises, the dropped side of the first substrate may contact a drop of reagent medium.

FIG. 3C shows a full closure of the sandwich between the first substrate and the second substrate with one or more spacers contacting both the first substrate and the second substrate.

FIG. 4A shows a side view of the angled closure workflow.

FIG. 4B shows a top view of the angled closure workflow.

FIG. 5 is a schematic diagram showing an example of a barcoded capture probe, as described herein.

FIG. 6 shows a schematic illustrating a cleavable capture probe.

FIG. 7 shows exemplary capture domains on capture probes.

FIG. 8 shows an exemplary arrangement of barcoded features within an array.

FIG. 9A shows an exemplary workflow for performing templated capture and producing a ligation product, and FIG. 9B shows an exemplary workflow for capturing a ligation product from FIG. 9A on a substrate.

FIG. 10 is a schematic diagram of an exemplary analyte capture agent.

FIG. 11 is a schematic diagram depicting an exemplary interaction between a feature-immobilized capture probe 1124 and an analyte capture agent 1126.

FIGS. 12A-D are representative images of the output of a spatial transcriptomics assay where a biological sample exceeded the boundaries of the capture probes on the array. Distortion of the signal for capture probes on the periphery of the array, i.e., “hot edges,” are indicated with white arrows.

FIG. 13A is an exemplary cross-section schematic of a biological sample including analytes disposed on a substrate and aligned with an array comprising capture probes, where a portion of the biological sample exceeds the boundaries of the capture probes on the array.

FIG. 13B is an exemplary cross-section schematic of a biological sample disposed on a substrate and aligned with an array comprising capture probes, where a portion of the tissue sample exceeds the boundaries of the capture probes on the array and analytes have migrated from the biological sample to the array.

FIG. 13C is an exemplary cross-section schematic of a biological sample disposed on a substrate and aligned with an array comprising capture probes, where a portion of the tissue example exceeds the boundaries of the capture probes on the array. Analytes have migrated from the biological sample to the array, however, some of the analytes that migrated from the portion of the biological sample that exceeds the boundaries of the capture probes on the array are mislocalized and captured by capture probes at the periphery of the array.

FIG. 14 is an exemplary schematic of capture probes on an array. Capture probes interior to the array, representing a majority of the capture probes and depicted in white, can include capture domains and can include spatial barcodes, unique molecular identifiers (UMIs), and/or functional domains as exemplified in FIG. 5 and FIG. 9B. Capture probes at the boundaries, or periphery, of the array, depicted in gray, include capture domains but do not include spatial barcodes or functional sequencing domains.

FIG. 15A is an exemplary cross-section schematic of a biological sample including analytes disposed on a substrate and aligned with an array comprising capture probes. The array includes capture probes at the interior of the array, depicted in white, which include capture domains and spatial barcodes, unique molecular identifiers (UMIs), and/or functional domains as exemplified in FIG. 5 and FIG. 9B, and capture probes at the boundaries of the array, depicted in gray, which include capture domains but do not include spatial barcodes or functional sequencing domains.

FIG. 15B is an exemplary cross-section schematic of a biological sample disposed on a substrate and aligned with an array comprising capture probes. The array includes capture probes at the interior of the array, depicted in white, which include capture domains and can include spatial barcodes, unique molecular identifiers (UMIs), and/or functional domains as exemplified in FIG. 5 and FIG. 9B, and capture probes at the boundaries of the array, depicted in gray, which include capture domains but do not include spatial barcodes or functional sequencing domains. The tissue sample overlaps both regions of capture probes. Analytes have migrated from the biological sample to the capture probes on the array. Analytes that migrated from the portion of the biological sample overlapping the capture probes depicted in white, which include capture domains and can include spatial barcodes, unique molecular identifiers (UMIs), and/or functional domains, are expected to be accurately detected by the spatial analysis and generate spatially related data. Analytes that migrated from the portion of the biological sample overlapping the capture probes at the boundaries of the array, depicted in gray, which include capture domains but do not include spatial barcodes or functional sequencing domains, will be captured but will not generate data, thereby reducing the effect of mislocalization of analytes on spatially related data from the biological sample.

### DETAILED DESCRIPTION

### A. Spatial Analysis Methods

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample. Intermediate agents (e.g., ligation products or other sequences) can serve as proxies of target analytes in the methods and compositions herein.

Non-limiting aspects of spatial analysis methodologies and compositions are described in U.S. Pat. Nos. 11,447,807, 11,352,667, 11,168,350, 11,104,936, 11,008,608, 10,995,361, 10,913,975, 10,774,374, 10,724,078, 10,640,816, 10,494,662, 10,480,022, 10,364,457, 10,317,321, 10,059,990, 10,041,949, 10,030,261, 10,002,316, 9,879,313, 9,783,841, 9,727,810, 9,593,365, 8,951,726, 8,604,182, and 7,709,198; U.S. Patent Application Publication Nos. 2020/0239946, 2020/0080136, 2020/0277663, 2019/0330617, 2020/0256867, 2020/0224244, 2019/0085383, and 2013/0171621; PCT Publication Nos. WO2018/091676, WO2020/176788, WO2017/144338, and WO2016/057552; Non-patent literature references Rodriques et al., Science 363(6434): 1463-1467, 2019; Lee et al., Nat. Protoc. 10(3): 442-458, 2015; Trejo et al., PLOS ONE 14(2): e0212031, 2019; Chen et al., Science 348(6233): aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev F, dated January 2022); and/or the Visium Spatial Gene Expression Reagent Kits-Tissue Optimization User Guide (e.g., Rev E, dated February 2022), both of which are available at the 10× Genomics Support Documentation website, and can be used herein in any combination, and each of which is incorporated herein by reference in their entireties. Further non-limiting aspects of spatial analysis methodologies and compositions are described herein.

Some general terminology that may be used in this disclosure can be found in Section (I)(b) of PCT Publication No. WO2020/176788 and/or U.S. Patent Application Publication No. 2020/0277663. Typically, a “barcode” is a label, or identifier, which conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of nucleic acid analytes include, but are not limited to, DNA, RNA (e.g., mRNA), and combinations thereof. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of PCT Publication No. WO2020/176788 and/or U.S. Patent Application Publication No. 2020/0277663. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, the biological sample is a tissue sample. In some embodiments, the biological sample (e.g., tissue sample) is a tissue microarray (TMA). A tissue microarray contains multiple representative tissue samples-which can be from different tissues or organisms-assembled on a single histologic slide. The TMA can therefore allow for high throughput analysis of multiple specimens at the same time. Tissue microarrays are paraffin blocks produced by extracting cylindrical tissue cores from different paraffin donor blocks and re-embedding these into a single recipient (microarray) block at defined array coordinates.

The biological sample as used herein can be any suitable biological sample described herein or known in the art. In some embodiments, the biological sample is a tissue. In some embodiments, the tissue sample is a solid tissue sample. In some embodiments, the biological sample is a tissue section. In some embodiments, the tissue is flash-frozen and sectioned. Any suitable method described herein or known in the art can be used to flash-freeze and section the tissue sample. In some embodiments, the biological sample, e.g., the tissue, is flash-frozen using liquid nitrogen before sectioning. In some embodiments, the biological sample, e.g., a tissue sample, is flash-frozen using nitrogen (e.g., liquid nitrogen), isopentane, or hexane.

In some embodiments, the biological sample, e.g., the tissue, is embedded in a matrix e.g., optimal cutting temperature (OCT) compound to facilitate sectioning. OCT compound is a formulation of clear, water-soluble glycols and resins, providing a solid matrix to encapsulate biological (e.g., tissue) specimens. In some embodiments, the sectioning is performed using cryosectioning. In some embodiments, the methods further comprise a thawing step, after the cryosectioning.

The biological sample can be from a mammal. In some instances, the biological sample is from a human, mouse, or rat. In addition to the subjects described above, the biological sample can be obtained from non-mammalian organisms (e.g., a plants, an insect, an arachnid, a nematode (e.g., Caenorhabditis elegans), a fungi, an amphibian, or a fish (e.g., zebrafish)). A biological sample can be obtained from a prokaryote such as a bacterium, e.g., Escherichia coli, Staphylococci or Mycoplasma pneumoniae; an archaea; a virus such as Hepatitis C virus or human immunodeficiency virus; or a viroid. A biological sample can be obtained from a eukaryote, such as a patient derived organoid (PDO) or patient derived xenograft (PDX). The biological sample can include organoids, a miniaturized and simplified version of an organ produced in vitro in three dimensions that shows realistic micro-anatomy. Organoids can be generated from one or more cells from a tissue, embryonic stem cells, and/or induced pluripotent stem cells, which can self-organize in three-dimensional culture owing to their self-renewal and differentiation capacities. In some embodiments, an organoid is a cerebral organoid, an intestinal organoid, a stomach organoid, a lingual organoid, a thyroid organoid, a thymic organoid, a testicular organoid, a hepatic organoid, a pancreatic organoid, an epithelial organoid, a lung organoid, a kidney organoid, a gastruloid, a cardiac organoid, or a retinal organoid. Subjects from which biological samples can be obtained can be healthy or asymptomatic individuals, individuals that have or are suspected of having a disease (e.g., cancer) or a pre-disposition to a disease, and/or individuals that are in need of therapy or suspected of needing therapy.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells.

In some embodiments, the biological sample, e.g., the tissue sample, is fixed in a fixative including alcohol, for example methanol. In some embodiments, instead of methanol, acetone, or an acetone-methanol mixture can be used. In some embodiments, the fixation is performed after sectioning. In some instances, the biological sample is not fixed with paraformaldehyde (PFA). In some instances, when the biological sample is fixed with a fixative including an alcohol (e.g., methanol or acetone-methanol mixture), it is not decrosslinked afterward. In some preferred embodiments, the biological sample is fixed with a fixative including an alcohol (e.g., methanol or an acetone-methanol mixture) after freezing and/or sectioning. In some instances, the biological sample is flash-frozen, and then the biological sample is sectioned and fixed (e.g., using methanol, acetone, or an acetone-methanol mixture). In some instances when methanol, acetone, or an acetone-methanol mixture is used to fix the biological sample, the sample is not decrosslinked at a later step. In instances when the biological sample is frozen (e.g., flash frozen using liquid nitrogen and embedded in OCT) followed by sectioning and alcohol (e.g., methanol, acetone-methanol) fixation or acetone fixation, the biological sample is referred to as “fresh frozen.” In some embodiments, fixation of the biological sample e.g., using acetone and/or alcohol (e.g., methanol, acetone-methanol) is performed while the sample is mounted on a substrate (e.g., glass slide, such as a positively charged glass slide).

In some embodiments, the biological sample, e.g., the tissue sample, is fixed e.g., immediately after being harvested from a subject. In such embodiments, the fixative is preferably an aldehyde fixative, such as paraformaldehyde (PFA) or formalin. In some embodiments, the fixative induces crosslinks within the biological sample. In some embodiments, after fixing e.g., by formalin or PFA, the biological sample is dehydrated via sucrose gradient. In some instances, the fixed biological sample is treated with a sucrose gradient and then embedded in a matrix e.g., OCT compound. In some instances, the fixed biological sample is not treated with a sucrose gradient, but rather is embedded in a matrix e.g., OCT compound after fixation. In some embodiments when a fixed frozen tissue sample is treated with a sucrose gradient, it can be rehydrated with an ethanol gradient. In some embodiments, the PFA or formalin fixed biological sample, which can be optionally dehydrated via sucrose gradient and/or embedded in OCT compound, is then frozen e.g., for storage or shipment. In such instances, the biological sample is referred to as “fixed frozen”. In preferred embodiments, a fixed frozen biological sample is not treated with methanol. In preferred embodiments, a fixed frozen biological sample is not paraffin embedded. Thus, in preferred embodiments, a fixed frozen biological sample is not deparaffinized. In some embodiments, a fixed frozen biological sample is rehydrated in an ethanol gradient.

In some instances, the biological sample (e.g., a fixed frozen tissue sample) is treated with a citrate buffer. Citrate buffer can be used for antigen retrieval to decrosslink antigens and fixation medium in the biological sample. Thus, any suitable decrosslinking agent can be used in addition to or alternatively to citrate buffer. In some embodiments, for example, the biological sample (e.g., a fixed frozen tissue sample) is decrosslinked with TE buffer.

In any of the foregoing, the biological sample can further be stained, imaged, and/or destained. For example, in some embodiments, a fresh frozen tissue sample or fixed frozen tissue sample is stained (e.g., via eosin and/or hematoxylin), imaged, destained (e.g., via HCl), or a combination thereof. In some embodiments, when a fresh frozen tissue sample is fixed in methanol, it is treated with isopropanol prior to being stained (e.g., via eosin and/or hematoxylin), imaged, destained (e.g., via HCl), or a combination thereof. In some embodiments when a fixed frozen tissue sample is treated with a sucrose gradient, it can be rehydrated with an ethanol gradient before being stained, (e.g., via eosin and/or hematoxylin), imaged, destained (e.g., via HCl), decrosslinked (e.g., via TE buffer or citrate buffer), or a combination thereof. In some embodiments, the biological sample can undergo further fixation (e.g., while mounted on a substrate), stained, imaged, and/or destained. For example, a fixed frozen biological sample may be subject to an additional fixing step (e.g., using PFA) before optional ethanol rehydration, staining, imaging, and/or destaining.

In any of the foregoing, the biological sample can be fixed using PAXgene. For example, the biological sample can be fixed using PAXgene in addition, or alternatively to, a fixative disclosed herein or known in the art (e.g., alcohol, acetone, acetone-alcohol, formalin, paraformaldehyde). PAXgene is a non-cross-linking mixture of different alcohols, acid and a soluble organic compound that preserves morphology and bio-molecules. It is a two-reagent fixative system in which tissue is firstly fixed in a solution containing methanol and acetic acid then stabilized in a solution containing ethanol. See, Ergin B. et al., J Proteome Res. 2010 Oct. 1; 9(10):5188-96; Kap M. et al., PLOS One.; 6(11):e27704 (2011); and Mathieson W. et al., Am J Clin Pathol.; 146 (1): 25-40 (2016), each of which are hereby incorporated by reference in their entirety, for a description and evaluation of PAXgene for tissue fixation. Thus, in some embodiments, when the biological sample, e.g., the tissue sample, is fixed in a fixative including alcohol, the fixative is PAXgene. In some embodiments, a fresh frozen tissue sample is fixed with PAXgene. In some embodiments, a fixed frozen tissue sample is fixed with PAXgene.

In some embodiments, the biological sample, e.g., the tissue sample is fixed, for example in methanol, acetone, acetone-methanol, PFA, PAXgene or is formalin-fixed and paraffin-embedded (FFPE). In some embodiments, the biological sample comprises intact cells. In some embodiments, the biological sample is a cell pellet, e.g., a fixed cell pellet, e.g., an FFPE cell pellet. FFPE samples are used in some instances in the RTL methods disclosed herein. A limitation of direct RNA capture for fixed samples is that the RNA integrity of fixed (e.g., FFPE) samples can be lower than a fresh sample, thereby making it more difficult to capture RNA directly, e.g., by capture of a common sequence such as a poly(A) tail of an mRNA molecule. However, by utilizing RTL probes that hybridize to RNA target sequences in the transcriptome, one can avoid a requirement for RNA analytes to have both a poly(A) tail and target sequences intact. Accordingly, RTL probes can be utilized to beneficially improve capture and spatial analysis of fixed samples. The biological sample, e.g., tissue sample, can be stained, and imaged prior, during, and/or after each step of the methods described herein. Any of the methods described herein or known in the art can be used to stain and/or image the biological sample. In some embodiments, the imaging occurs prior to destaining the sample. In some embodiments, the biological sample is stained using an H&E staining method. In some embodiments, the tissue sample is stained and imaged for about 10 minutes to about 2 hours (or any of the subranges of this range described herein). Additional time may be needed for staining and imaging of different types of biological samples.

The tissue sample can be obtained from any suitable location in a tissue or organ of a subject, e.g., a human subject. In some instances, the sample is a mouse sample. In some instances, the sample is a human sample. In some embodiments, the sample can be derived from skin, brain, breast, lung, liver, kidney, prostate, tonsil, thymus, testes, bone, lymph node, ovary, eye, heart, or spleen. In some instances, the sample is a human or mouse breast tissue sample. In some instances, the sample is a human or mouse brain tissue sample. In some instances, the sample is a human or mouse lung tissue sample. In some instances, the sample is a human or mouse tonsil tissue sample. In some instances, the sample is a human or mouse liver tissue sample. In some instances, the sample is a human or mouse bone, skin, kidney, thymus, testes, or prostate tissue sample. In some embodiments, the tissue sample is derived from normal or diseased tissue. In some embodiments, the sample is an embryo sample. The embryo sample can be a non-human embryo sample. In some instances, the sample is a mouse embryo sample.

Non-limiting examples of stains include histological stains (e.g., hematoxylin and/or eosin) and immunological stains (e.g., fluorescent stains). The biological sample can be stained using Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS) staining techniques. In some instances, PAS staining is performed after formalin or acetone fixation. In some embodiments, a biological sample (e.g., a fixed and/or stained biological sample) can be imaged. Biological samples are also described in Section (I)(d) of PCT Publication No. WO2020/176788 and/or U.S. Patent Application Publication No. 2020/0277663.

The following embodiments can be used with any of the methods described herein. In some embodiments, the biological sample is imaged. In some embodiments, the biological sample is visualized or imaged using bright field microscopy. In some embodiments, the biological sample is visualized or imaged using fluorescence microscopy. Additional methods of visualization and imaging are known in the art. Non-limiting examples of visualization and imaging include expansion microscopy, bright field microscopy, dark field microscopy, phase contrast microscopy, electron microscopy, fluorescence microscopy, reflection microscopy, interference microscopy and confocal microscopy. In some embodiments, the sample is stained and imaged prior to adding the primer to the biological sample.

In some embodiments, the method includes staining the biological sample. In some embodiments, the staining includes the use of hematoxylin and eosin. In some embodiments, a biological sample can be stained using any number of biological stains, including but not limited to, acridine orange, Bismarck brown, carmine, coomassie blue, cresyl violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, or safranin. In some instances, the biological sample can be stained using known staining techniques, including Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS) staining techniques. PAS staining is typically performed after formalin or acetone fixation.

In some embodiments, the staining includes the use of a detectable label selected from the group consisting of a radioisotope, a fluorophore, a chemiluminescent compound, a bioluminescent compound, or a combination thereof.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of PCT Publication No. WO2020/176788 and/or U.S. Patent Application Publication No. 2020/0277663. Briefly, in any of the methods described herein, the method includes a step of permeabilizing the biological sample. For example, the biological sample can be permeabilized to facilitate transfer of the extension products to the capture probes on the array. In some embodiments, the permeabilizing includes the use of an organic solvent (e.g., acetone, ethanol, and methanol), a detergent (e.g., saponin, Triton X-100™, Tween-20™, or sodium dodecyl sulfate (SDS)), an enzyme (an endopeptidase, an exopeptidase, a protease), or combinations thereof. In some embodiments, the permeabilizing includes the use of an endopeptidase, a protease, SDS, polyethylene glycol tert-octylphenyl ether, polysorbate 80, and polysorbate 20, N-lauroylsarcosine sodium salt solution, saponin, Triton X-100™, Tween-20™, or combinations thereof. In some embodiments, the endopeptidase is pepsin. In some embodiments, the endopeptidase is Proteinase K. Additional methods for sample permeabilization are described, for example, in Jamur et al., Method Mol. Biol. 588:63-66, 2010, the entire contents of which are incorporated herein by reference.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some instances, the capture probe includes a homopolymer sequence, such as a poly(T) sequence. In some embodiments, a capture probe can include a cleavage domain and/or a functional domain (e.g., a primer-binding site, such as for next-generation sequencing (NGS)). See, e.g., Section (II)(b) (e.g., subsections (i)-(vi)) of PCT Publication No. WO2020/176788 and/or U.S. Patent Application Publication No. 2020/0277663. Generation of capture probes can be achieved by any appropriate method, including those described in Section (II)(d)(ii) of PCT Publication No. WO2020/176788 and/or U.S. Patent Application Publication No. 2020/0277663.

In some instances, a capture probe and a nucleic acid analyte (or any other nucleic acid to nucleic acid interaction) occurs because the sequences of the two nucleic acids are substantially complementary to one another. By “substantial,” “substantially” and the like, two nucleic acid sequences can be complementary when at least 60% of the nucleotide residues of one nucleic acid sequence are complementary to nucleotide residues in the other nucleic acid sequence. The complementary residues within a particular complementary nucleic acid sequence need not always be contiguous with each other and can be interrupted by one or more non-complementary residues within the complementary nucleic acid sequence. In some embodiments, at least 60%, but less than 100%, of the residues of one of the two complementary nucleic acid sequences are complementary to residues in the other nucleic acid sequence. In some embodiments, at least 70%, 80%, 90%, 95% or 99% of the residues of one nucleic acid sequence are complementary to residues in the other nucleic acid sequence. Sequences are said to be “substantially complementary” when at least 60% (e.g., at least 70%, at least 80%, or at least 90%) of the residues of one nucleic acid sequence are complementary to residues in the other nucleic acid sequence.

In some embodiments, more than one analyte type (e.g., nucleic acids and proteins) from a biological sample can be detected (e.g., simultaneously or sequentially) using any appropriate multiplexing technique, such as those described in Section (IV) of PCT Publication No. WO2020/176788 and/or U.S. Patent Application Publication No. 2020/0277663.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) an analyte capture sequence. As used herein, the term “analyte binding moiety barcode” refers to a barcode that is associated with or otherwise identifies the analyte binding moiety. As used herein, the term “analyte capture sequence” refers to a region or moiety configured to hybridize to, bind to, couple to, or otherwise interact with a capture domain of a capture probe. In some cases, an analyte binding moiety barcode (or portion thereof) may be able to be removed (e.g., cleaved) from the analyte capture agent. Additional description of analyte capture agents can be found in Section (II)(b)(ix) of PCT Publication No. WO2020/176788 and/or Section (II)(b)(viii) U.S.

### Patent Application Publication No. 2020/0277663.

In some embodiments, the biological sample is mounted on a first substrate and the substrate comprising the array of capture probes is a second substrate. During this process, one or more analytes or analyte derivatives (e.g., intermediate agents; e.g., ligation products) are released from the biological sample and migrate to the second substrate comprising an array of capture probes. In some embodiments, the release and migration of the analytes or analyte derivatives to the second substrate comprising the array of capture probes occurs in a manner that preserves the original spatial context of the analytes in the biological sample. This method can be referred to as a sandwiching process, which is described e.g., in U.S. Patent Application Pub. No. 2021/0189475 and PCT Pub. Nos. WO 2021/252747 A1, WO 2022/061152 A2, and WO 2022/140028 A1.

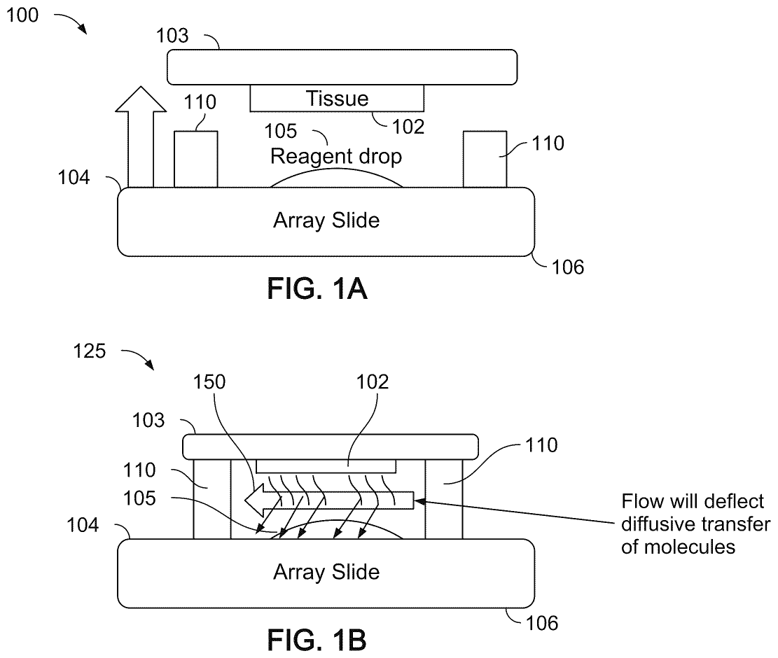

FIG. 1A shows an exemplary sandwiching process 100 where a first substrate (e.g., slide 103), including a biological sample 102 (e.g., a tissue section), and a second substrate (e.g., array slide 104 including an array having spatially barcoded capture probes 106) are brought into proximity with one another. As shown in FIG. 1A a liquid reagent drop (e.g., permeabilization solution 105) is introduced on the second substrate in proximity to the capture probes 106 and in between the biological sample 102 and the second substrate (e.g., slide 104 including an array having spatially barcoded capture probes 106). The permeabilization solution 105 may release analytes or analyte derivatives (e.g., intermediate agents; e.g., ligation products) that can be captured by the capture probes of the array 106.

During the exemplary sandwiching process, the first substrate is aligned with the second substrate, such that at least a portion of the biological sample is aligned with at least a portion of the capture probes (e.g., aligned in a sandwich configuration). As shown, the second substrate (e.g., array slide 104) is in an inferior position to the first substrate (e.g., slide 103). In some embodiments, the first substrate (e.g., slide 103) may be positioned superior to the second substrate (e.g., slide 104). A reagent medium 105 within a gap between the first substrate (e.g., slide 103) and the second substrate (e.g., slide 104) creates a liquid interface between the two substrates. The reagent medium may be a permeabilization solution which permeabilizes and/or digests the biological sample 102. In some embodiments wherein the biological sample 102 has been pre-permeabilized, the reagent medium is not a permeabilization solution. In some embodiments, analytes (e.g., mRNA transcripts) and/or analyte derivatives (e.g., intermediate agents; e.g., ligation products) of the biological sample 102 may release from the biological sample, and actively or passively migrate (e.g., diffuse) across the gap toward the capture probes on the array 106. Alternatively, in certain embodiments, migration of the analyte or analyte derivative (e.g., intermediate agent; e.g., ligation product) from the biological sample is performed actively (e.g., electrophoretic, by applying an electric field to promote migration). Exemplary methods of electrophoretic migration are described in WO 2020/176788, and US. Patent Application Pub. No. 2021/0189475, each of which is hereby incorporated by reference.

As further shown, one or more spacers 110 may be positioned between the first substrate (e.g., slide 103) and the second substrate (e.g., array slide 104 including spatially barcoded capture probes 106). The one or more spacers 110 may be configured to maintain a separation distance between the first substrate and the second substrate. While the one or more spacers 110 is shown as disposed on the second substrate, the spacer may additionally or alternatively be disposed on the first substrate.

In some embodiments, the one or more spacers 110 is configured to maintain a separation distance between first and second substrates that is between about 2 microns and 1 mm (e.g., between about 2 microns and 800 microns, between about 2 microns and 700 microns, between about 2 microns and 600 microns, between about 2 microns and 500 microns, between about 2 microns and 400 microns, between about 2 microns and 300 microns, between about 2 microns and 200 microns, between about 2 microns and 100 microns, between about 2 microns and 25 microns, or between about 2 microns and 10 microns), measured in a direction orthogonal to the surface of first substrate that supports the biological sample. In some instances, the separation distance is about 2, 3, 4, 5, 6, 7, 8, 9, 10, 11, 12, 13, 14, 15, 16, 17, 18, 19, 20, 21, 22, 23, 24, or 25 microns. In some embodiments, the separation distance is less than 50 microns. In some embodiments, the separation distance is less than 25 microns. In some embodiments, the separation distance is less than 20 microns. The separation distance may include a distance of at least 2 μm.

FIG. 1B shows a fully formed sandwich configuration 125 creating a chamber 150 formed from the one or more spacers 110, the first substrate (e.g., the slide 103), and the second substrate (e.g., the slide 104 including an array 106 having spatially barcoded capture probes) in accordance with some example implementations. In the example of FIG. 1B, the liquid reagent (e.g., the permeabilization solution 105) fills the volume of the chamber 150 and may create a permeabilization buffer that allows analytes (e.g., mRNA transcripts and/or other molecules) or analyte derivatives (e.g., intermediate agents; e.g., ligation products) to diffuse from the biological sample 102 toward the capture probes of the second substrate (e.g., slide 104). In some aspects, flow of the permeabilization buffer may deflect transcripts and/or molecules from the biological sample 102 and may affect diffusive transfer of analytes or analyte derivatives (e.g., intermediate agents; e.g., ligation products) for spatial analysis. A partially or fully sealed chamber 150 resulting from the one or more spacers 110, the first substrate, and the second substrate may reduce or prevent flow from undesirable convective movement of transcripts and/or molecules over the diffusive transfer from the biological sample 102 to the capture probes.

The sandwiching process methods described above can be implemented using a variety of hardware components. For example, the sandwiching process methods can be implemented using a sample holder (also referred to herein as a support device, a sample handling apparatus, and an array alignment device). Further details on support devices, sample holders, sample handling apparatuses, or systems for implementing a sandwiching process are described in, e.g., US. Patent Application Pub. No. 2021/0189475, and PCT Publ. No. WO 2022/061152 A2, each of which are incorporated by reference in their entirety.

In some embodiments of a sample holder, the sample holder can include a first member including a first retaining mechanism configured to retain a first substrate comprising a biological sample. The first retaining mechanism can be configured to retain the first substrate disposed in a first plane. The sample holder can further include a second member including a second retaining mechanism configured to retain a second substrate disposed in a second plane. The sample holder can further include an alignment mechanism connected to one or both of the first member and the second member. The alignment mechanism can be configured to align the first and second members along the first plane and/or the second plane such that the sample contacts at least a portion of the reagent medium when the first and second members are aligned and within a threshold distance along an axis orthogonal to the second plane. The adjustment mechanism may be configured to move the second member along the axis orthogonal to the second plane and/or move the first member along an axis orthogonal to the first plane.

In some embodiments, the adjustment mechanism includes a linear actuator. In some embodiments, the linear actuator is configured to move the second member along an axis orthogonal to the plane of the first member and/or the second member. In some embodiments, the linear actuator is configured to move the first member along an axis orthogonal to the plane of the first member and/or the second member. In some embodiments, the linear actuator is configured to move the first member, the second member, or both the first member and the second member at a velocity of at least 0.1 mm/sec. In some embodiments, the linear actuator is configured to move the first member, the second member, or both the first member and the second member with an amount of force of at least 0.1 lbs.

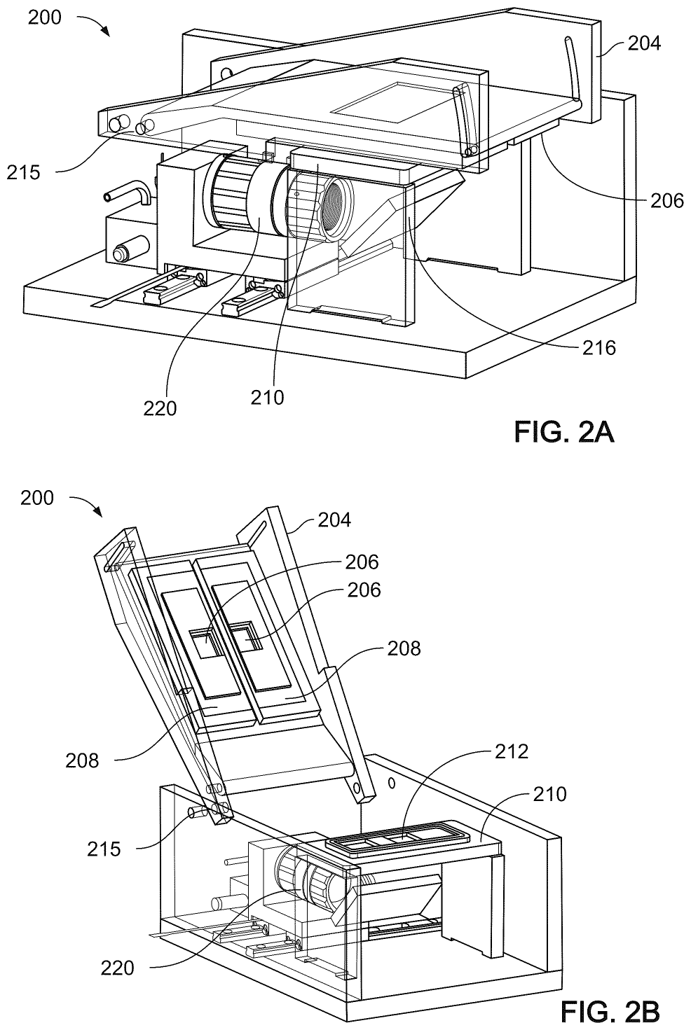

FIG. 2A is a perspective view of an example sample handling apparatus 200 in a closed position in accordance with some example implementations. As shown, the sample handling apparatus 200 includes a first member 204, a second member 210, optionally an image capture device 220, a first substrate 206, optionally a hinge 215, and optionally a mirror 216. The hinge 215 may be configured to allow the first member 204 to be positioned in an open or closed configuration by opening and/or closing the first member 204 in a clamshell manner along the hinge 215.

FIG. 2B is a perspective view of the example sample handling apparatus 200 in an open position in accordance with some example implementations. As shown, the sample handling apparatus 200 includes one or more first retaining mechanisms 208 configured to retain one or more first substrates 206. In the example of FIG. 2B, the first member 204 is configured to retain two first substrates 206, however the first member 204 may be configured to retain more or fewer first substrates 206.

In some aspects, when the sample handling apparatus 200 is in an open position (e.g., in FIG. 2B), the first substrate 206 and/or the second substrate 212 may be loaded and positioned within the sample handling apparatus 200 such as within the first member 204 and the second member 210, respectively. As noted, the hinge 215 may allow the first member 204 to close over the second member 210 and form a sandwich configuration.

In some aspects, after the first member 204 closes over the second member 210, an adjustment mechanism of the sample handling apparatus 200 may actuate the first member 204 and/or the second member 210 to form the sandwich configuration for the permeabilization step (e.g., bringing the first substrate 206 and the second substrate 212 closer to each other and within a threshold distance for the sandwich configuration). The adjustment mechanism may be configured to control a speed, an angle, a force, or the like of the sandwich configuration.

In some embodiments, the biological sample (e.g., sample 102 from FIG. 1A) may be aligned within the first member 204 (e.g., via the first retaining mechanism 208) prior to closing the first member 204 such that a desired region of interest of the sample is aligned with the barcoded array of the second substrate (e.g., the slide 104 from FIG. 1A), e.g., when the first and second substrates are aligned in the sandwich configuration. Such alignment may be accomplished manually (e.g., by a user) or automatically (e.g., via an automated alignment mechanism). After or before alignment, spacers may be applied to the first substrate 206 and/or the second substrate 212 to maintain a minimum spacing between the first substrate 206 and the second substrate 212 during sandwiching. In some aspects, the permeabilization solution (e.g., permeabilization solution 305) may be applied to the first substrate 206 and/or the second substrate 212. The first member 204 may then close over the second member 210 and form the sandwich configuration. Analytes or analyte derivatives (e.g., intermediate agents; e.g., ligation products) may be captured by the capture probes of the array and may be processed for spatial analysis.

In some embodiments, during the permeabilization step, the image capture device 220 may capture images of the overlap area between the biological sample and the capture probes on the array 106. If more than one first substrates 206 and/or second substrates 212 are present within the sample handling apparatus 200, the image capture device 220 may be configured to capture one or more images of one or more overlap areas.

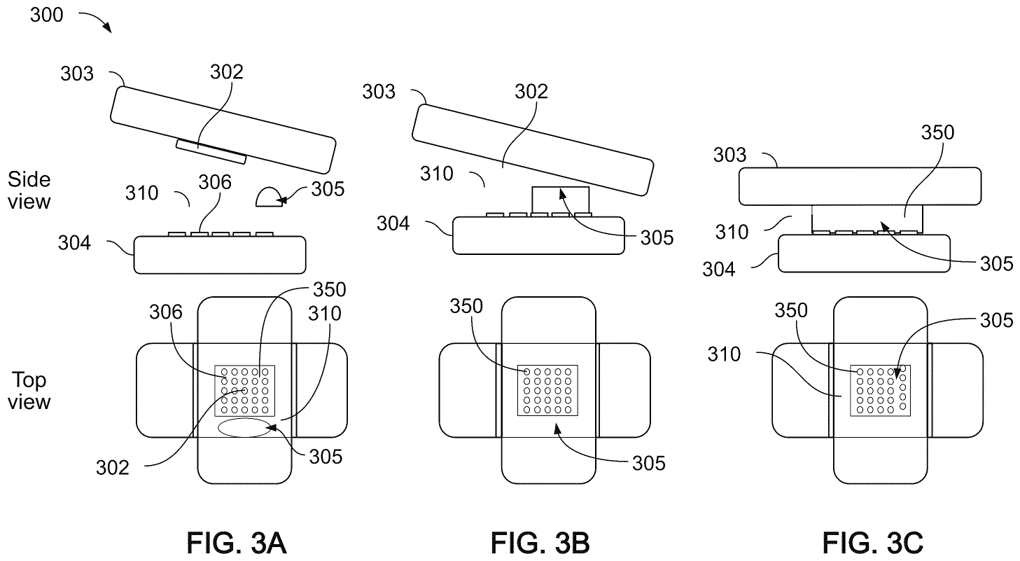

Provided herein are methods for delivering a fluid to a biological sample disposed on an area of a first substrate and an array disposed on a second substrate. FIGS. 3A-3C depict a side view and a top view of an exemplary angled closure workflow 300 for sandwiching a first substrate (e.g., slide 303) having a biological sample 302 and a second substrate (e.g., slide 304 having capture probes 306) in accordance with some exemplary implementations.

FIG. 3A depicts the first substrate (e.g., the slide 303 including a biological sample 302) angled over (superior to) the second substrate (e.g., slide 304). As shown, reagent medium (e.g., permeabilization solution) 305 is located on the spacer 310 toward the right-hand side of the side view in FIG. 3A. While FIG. 3A depicts the reagent medium on the right hand side of side view, it should be understood that such depiction is not meant to be limiting as to the location of the reagent medium on the spacer.

FIG. 3B shows that as the first substrate lowers, and/or as the second substrate rises, the dropped side of the first substrate (e.g., a side of the slide 303 angled toward the second substrate) may contact the reagent medium 305. The dropped side of the first substrate may urge the reagent medium 305 toward the opposite direction (e.g., towards an opposite side of the spacer 310, towards an opposite side of the first substrate relative to the dropped side). For example, in the side view of FIG. 3B the reagent medium 305 may be urged from right to left as the sandwich is formed.

In some embodiments, the first substrate and/or the second substrate are further moved to achieve an approximately parallel arrangement of the first substrate and the second substrate.

FIG. 3C depicts a full closure of the sandwich between the first substrate and the second substrate with the spacer 310 contacting both the first substrate and the second substrate and maintaining a separation distance and optionally the approximately parallel arrangement between the two substrates. As shown in the top view of FIG. 3C, the spacer 310 fully encloses and surrounds the biological sample 302 and the capture probes 306, and the spacer 310 form the sides of chamber 350 which holds a volume of the reagent medium 305.

While FIG. 3C depicts the first substrate (e.g., the slide 303 including biological sample 302) angled over (superior to) the second substrate (e.g., slide 304) and the second substrate comprising the spacer 310, it should be understood that an exemplary angled closure workflow can include the second substrate angled over (superior to) the first substrate and the first substrate comprising the spacer 310.

It may be desirable that the reagent medium be free from air bubbles between the substrates to facilitate transfer of target analytes with spatial information. Additionally, air bubbles present between the substrates may obscure at least a portion of an image capture of a desired region of interest. Accordingly, it may be desirable to ensure or encourage suppression and/or elimination of air bubbles between the two substrates (e.g., slide 303 and slide 304) during a permeabilization step (e.g., step 104). In some aspects, it may be possible to reduce or eliminate bubble formation between the substrates using a variety of filling methods and/or closing methods. In some instances, the first substrate and the second substrate are arranged in an angled sandwich assembly as described herein. For example, during the sandwiching of the two substrates (e.g., the slide 303 and the slide 304), an angled closure workflow may be used to suppress or eliminate bubble formation.

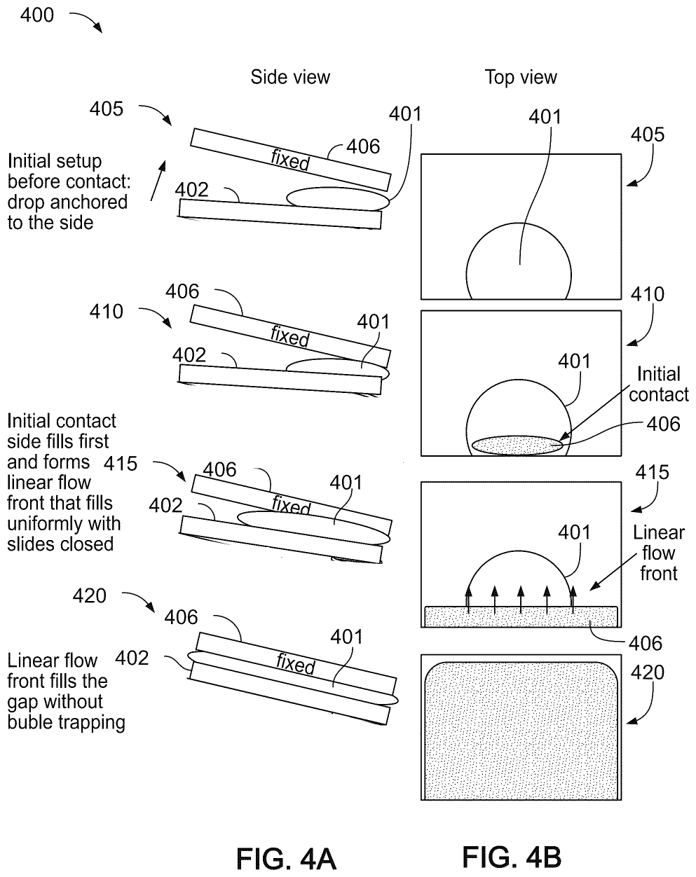

FIG. 4A is a side view of the angled closure workflow 400 in accordance with some exemplary implementations. FIG. 4B is a top view of the angled closure workflow 400 in accordance with some exemplary implementations. As shown at 405, reagent medium 401 is positioned to the side of the substrate 402.

At step 410, the dropped side of the angled substrate 406 contacts the reagent medium 401 first. The contact of the substrate 406 with the reagent medium 401 may form a linear or low curvature flow front that fills uniformly with the slides closed.

At step 415, the substrate 406 is further lowered toward the substrate 402 (or the substrate 402 is raised up toward the substrate 406) and the dropped side of the substrate 406 may contact and may urge the reagent medium toward the side opposite the dropped side and creating a linear or low curvature flow front that may prevent or reduce bubble trapping between the substrates.

At step 420, the reagent medium 401 fills the gap between the substrate 406 and the substrate 402. The linear flow front of the liquid reagent may form by squeezing the 401 volume along the contact side of the substrate 402 and/or the substrate 406. Additionally, capillary flow may also contribute to filling the gap area.

In some embodiments, the reagent medium (e.g., 105 in FIG. 1A) comprises a permeabilization agent. In some embodiments, following initial contact between the biological sample and a permeabilization agent, the permeabilization agent can be removed from contact with the biological sample (e.g., by opening sample holder). Suitable agents for this purpose include, but are not limited to, organic solvents (e.g., acetone, ethanol, and methanol), cross-linking agents (e.g., paraformaldehyde), detergents (e.g., saponin, Triton X-100™, Tween-20™, or sodium dodecyl sulfate (SDS)), and enzymes (e.g., trypsin, proteases (e.g., proteinase K). In some embodiments, the detergent is an anionic detergent (e.g., SDS or N-lauroylsarcosine sodium salt solution).

In some embodiments, the reagent medium comprises a lysis reagent. Lysis solutions can include ionic surfactants such as, for example, sarkosyl and sodium dodecyl sulfate (SDS). More generally, chemical lysis agents can include, without limitation, organic solvents, chelating agents, detergents, surfactants, and chaotropic agents. In some embodiments, the reagent medium comprises a protease. Exemplary proteases include, e.g., pepsin, trypsin, pepsin, elastase, and proteinase K. In some embodiments, the reagent medium comprises a nuclease. In some embodiments, the nuclease comprises an RNase. In some embodiments, the RNase is selected from RNase A, RNase C, RNase H, and RNase I. In some embodiments, the reagent medium comprises one or more of sodium dodecyl sulfate (SDS) or a sodium salt thereof, proteinase K, pepsin, N-lauroylsarcosine, and RNase.

In some embodiments, the reagent medium comprises polyethylene glycol (PEG). In some embodiments, the PEG is from about PEG 2K to about PEG 16K. In some embodiments, the PEG is PEG 2K, 3K, 4K, 5K, 6K, 7K, 8K, 9K, 10K, 11K, 12K, 13K, 14K, 15K, or 16K. In some embodiments, the PEG is present at a concentration from about 2% to 25%, from about 4% to about 23%, from about 6% to about 21%, or from about 8% to about 20% (v/v).

In certain embodiments a dried permeabilization reagent is applied or formed as a layer on the first substrate or the second substrate or both prior to contacting the biological sample and the array. For example, a permeabilization reagent can be deposited in solution on the first substrate or the second substrate or both and then dried.

In some instances, the aligned portions of the biological sample and the array are in contact with the reagent medium for about 1 minute, about 5 minutes, about 10 minutes, about 12 minutes, about 15 minutes, about 18 minutes, about 20 minutes, about 25 minutes, about 30 minutes, about 36 minutes, about 45 minutes, or about an hour. In some instances, the aligned portions of the biological sample and the array are in contact with the reagent medium for about 1-60 minutes.

In some instances, the device is configured to control a temperature of the first and second substrates. In some embodiments, the temperature of the first and second members is lowered to a first temperature that is below room temperature.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II)(b)(vii) of PCT Publication No. WO2020/176788 and/or U.S. Patent Application Publication No. 2020/0277663 regarding extended capture probes). In some cases, capture probes may be configured to form ligation products with a template (e.g., a DNA or RNA template, such as an analyte or an intermediate agent, or portion thereof), thereby creating ligations products that serve as proxies for the template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended by a reverse transcriptase. In some embodiments, the capture probe is extended using one or more DNA polymerases. In some embodiments, the extended capture probes include the sequence of the capture domain and the sequence of the spatial barcode of the capture probe.

In some embodiments, extended capture probes are amplified (e.g., in bulk solution or on the array) to yield quantities that are sufficient for downstream analysis, e.g., sequencing. In some embodiments, extended capture probes (e.g., DNA molecules) can act as templates for an amplification reaction (e.g., a polymerase chain reaction).

Additional variants of spatial analysis methods, including in some embodiments, an imaging step, are described in Section (II)(a) of PCT Publication No. WO2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of PCT Publication No. WO2020/176788 and/or U.S. Patent Application Publication No. 2020/0277663. Some quality control measures are described in Section (II)(h) of PCT Publication No. WO2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder. Exemplary methods for identifying spatial information of biological and/or medical importance can be found in U.S. Patent Application Publication Nos. 2021/0140982, 2021/0198741, and/or 2021/0199660.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor or proximity based analysis); determination of up- and/or down-regulated genes and/or proteins in diseased tissue; characterization of tumor microenvironments; characterization of tumor immune responses; characterization of cells types and their co-localization in healthy and diseased tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of PCT Publication No. WO2020/176788 and/or U.S. Patent Application Publication No. 2020/0277663. Exemplary features and geometric attributes of an array can be found in Sections (II)(d)(i), (II)(d)(iii), and (II)(d)(iv) of PCT Publication No. WO2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells, spots) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of PCT Publication No. WO2020/176788 and/or U.S. Patent Application Publication No. 2020/0277663.

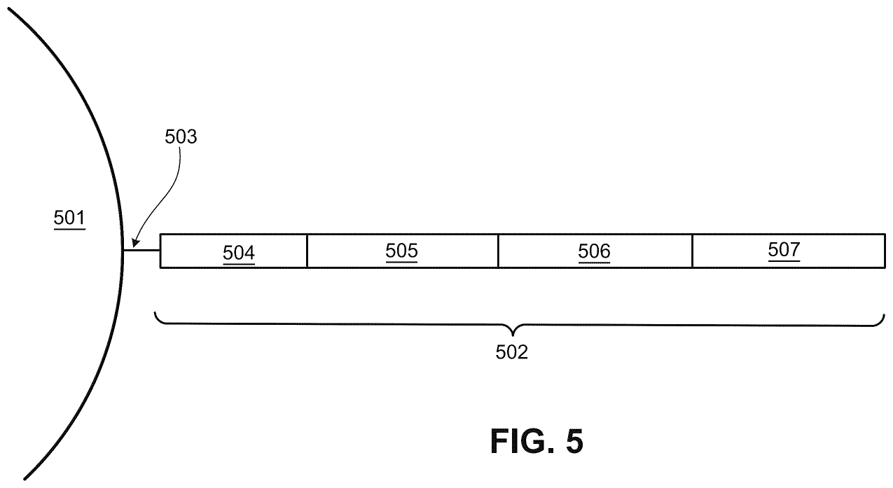

FIG. 5 is a schematic diagram showing an exemplary capture probe, as described herein. As shown, the capture probe 502 is optionally coupled to a feature 501 by a cleavage domain 503, such as a disulfide linker. The capture probe can include a functional sequence 504 that are useful for subsequent processing. The functional sequence 504 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 505. The capture probe can also include a unique molecular identifier (UMI) sequence 506. While FIG. 5 shows the spatial barcode 505 as being located upstream (5′) of UMI sequence 506, it is to be understood that capture probes wherein UMI sequence 506 is located upstream (5′) of the spatial barcode 505 is also suitable for use in any of the methods described herein. The capture probe can also include a capture domain 507 to facilitate capture of a target analyte. The capture domain can have a sequence complementary to a sequence of a nucleic acid analyte. The capture domain can have a sequence complementary to a connected probe described herein. The capture domain can have a sequence complementary to a capture handle sequence present in an analyte capture agent. The capture domain can have a sequence complementary to a splint oligonucleotide. Such splint oligonucleotide, in addition to having a sequence complementary to a capture domain of a capture probe, can have a sequence complementary to a sequence of a nucleic acid analyte, a portion of a connected probe described herein, a capture handle sequence described herein, and/or a methylated adaptor described herein.

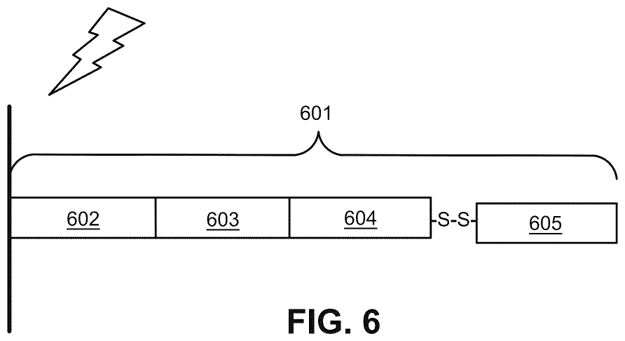

FIG. 6 is a schematic illustrating a cleavable capture probe, wherein the cleaved capture probe can enter into a cell and bind to analytes within the sample. The capture probe 601 contains a cleavage domain 602, a cell penetrating peptide 603, a reporter molecule 604, and a disulfide bond (—S—S—). 605 represents all other parts of a capture probe, for example a spatial barcode and a capture domain.

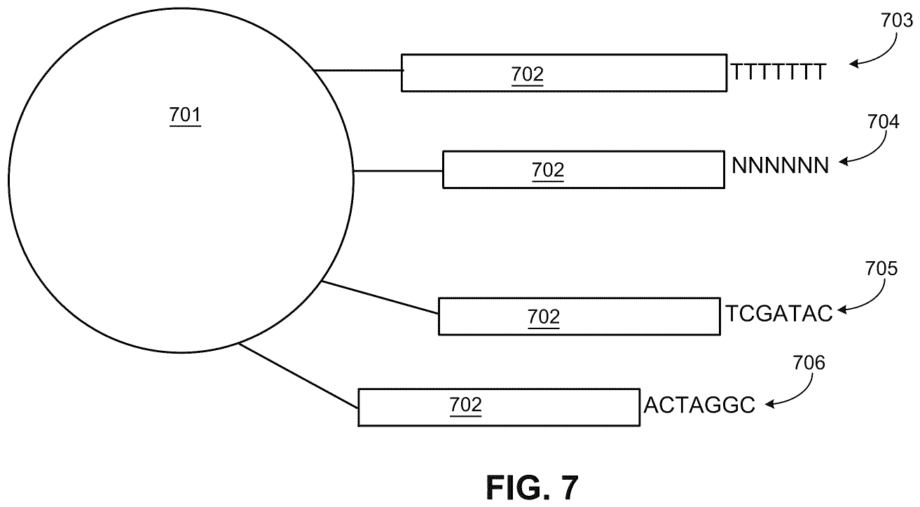

FIG. 7 is a schematic diagram of an exemplary multiplexed spatially-barcoded feature. In FIG. 7, the feature 701 (e.g., bead, well, spot) can be coupled to spatially-barcoded capture probes. In some embodiments, the spatially-barcoded probes of a particular feature can possess the same spatial barcode and the same capture domain. In some embodiments, the spatially-barcoded probes of a particular feature can possess the same spatial barcode but have different capture domains designed to associate the spatial barcode of the feature with more than one target analyte. For example, a feature may be coupled to four different types of spatially-barcoded capture probes, each type of spatially-barcoded capture probe possessing the spatial barcode 702. One type of capture probe associated with the feature includes the spatial barcode 702 in combination with a poly(T) capture domain 703, designed to capture mRNA target analytes. A second type of capture probe associated with the feature includes the spatial barcode 702 in combination with a random N-mer capture domain 704 for gDNA analysis. A third type of capture probe associated with the feature includes the spatial barcode 702 in combination with a capture domain complementary to the analyte capture agent of interest 705. A fourth type of capture probe associated with the feature includes the spatial barcode 702 in combination with a capture probe that can specifically bind a nucleic acid molecule 706 that can function in a CRISPR assay (e.g., CRISPR/Cas9). While only four different capture probe-barcoded constructs are shown in FIG. 7, capture-probe barcoded constructs can be tailored for analyses of any given analyte associated with a nucleic acid and capable of binding with such a construct. For example, the schemes shown in FIG. 7 can also be used for concurrent analysis of other analytes disclosed herein, including, but not limited to: (a) mRNA, a lineage tracing construct, cell surface or intracellular proteins and metabolites, and gDNA; (b) mRNA, accessible chromatin (e.g., ATAC-seq, DNase-seq, and/or MNase-seq) cell surface or intracellular proteins and metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V (D) J sequence of an immune cell receptor (e.g., T-cell receptor). In some embodiments, a perturbation agent can be a small molecule, an antibody, a drug, an aptamer, a miRNA, a physical environmental (e.g., temperature change), or any other known perturbation agents.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 505 and functional sequences 504 is common to all of the probes attached to a given feature. In some embodiments, the UMI sequence 506 of a capture probe attached to a given feature is different from the UMI sequence of a different capture probe attached to the given feature.

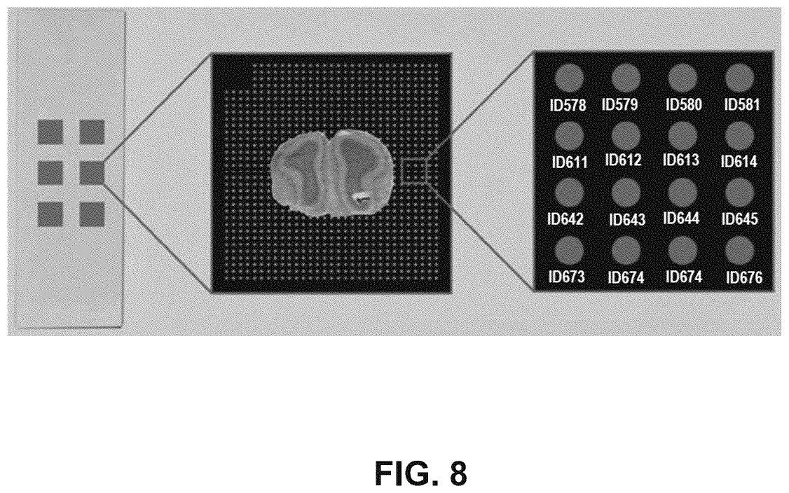

FIG. 8 depicts an exemplary arrangement of barcoded features within an array. From left to right, FIG. 8 shows (L) a slide including six spatially-barcoded arrays, (C) an enlarged schematic of one of the six spatially-barcoded arrays, showing a grid of barcoded features in relation to a biological sample, and (R) an enlarged schematic of one section of an array, Showing the specific identification of multiple features within the array (labelled as ID578, ID579, ID560, etc.).

In some embodiments, more than one analyte type (e.g., nucleic acids and proteins) from a biological sample can be detected (e.g., simultaneously or sequentially) using any appropriate multiplexing technique, such as those described in Section (IV) of PCT Publication No. WO2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of PCT Publication No. WO2020/176788 and/or U.S. Patent Application Publication No. 2020/0277663.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21; 45(14):e128. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture binding capture domain (e.g., a poly(A) sequence, a non-homopolymeric sequence). After hybridization to the analyte, a ligase (e.g., a T4 RNA ligase (Rnl2), a PBCV-1 DNA Ligase or Chlorella virus DNA Ligase, a single-stranded DNA ligase, or a T4 DNA ligase) ligates the two oligonucleotides together, creating a ligation product. In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNase H). In some instances, the ligation product is removed using heat. In some instances, the ligation product is removed using KOH. The released ligation product can then be captured by capture probes (e.g., instead of direct capture of an analyte) on an array, optionally amplified, and sequenced, thus determining the location and optionally the abundance of the analyte in the biological sample.

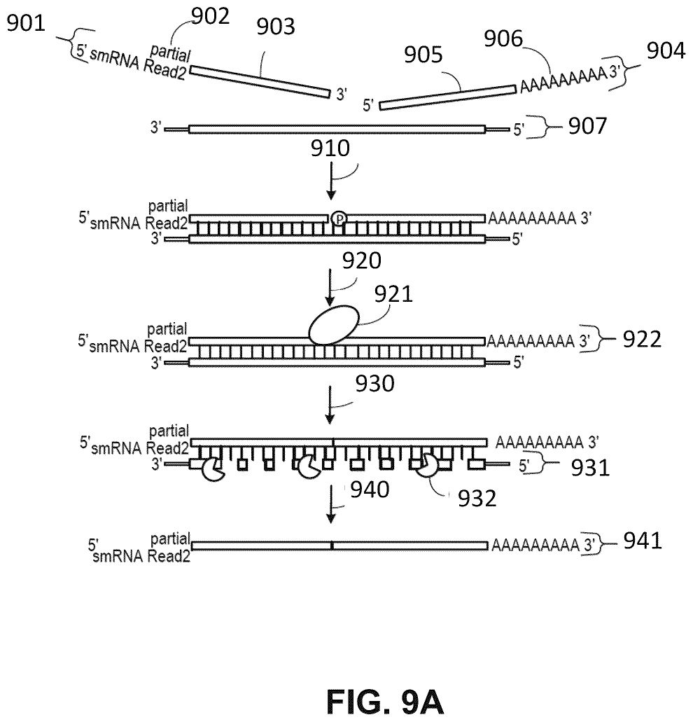

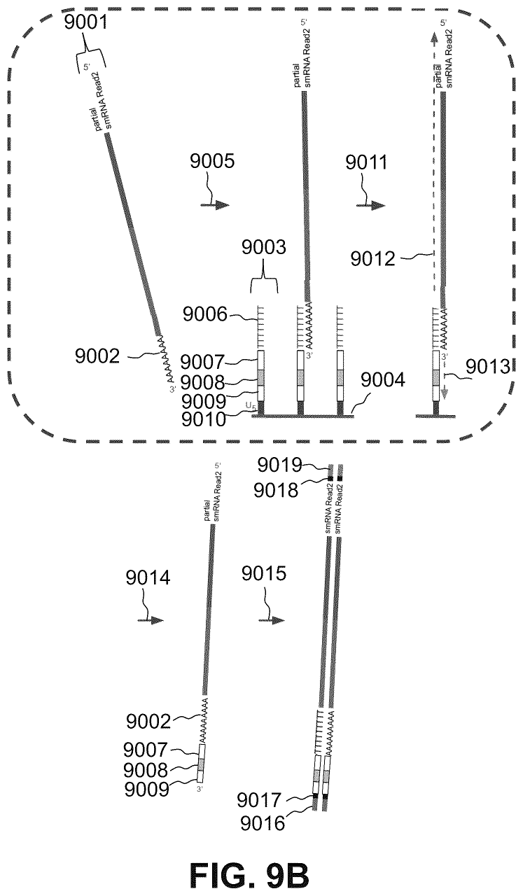

A non-limiting example of templated ligation methods disclosed herein is depicted in FIG. 9A. After a biological sample is contacted with a substrate including a plurality of capture probes and contacted with (a) a first probe 901 having a target-hybridization sequence 903 and a primer sequence 902 and (b) a second probe 904 having a target-hybridization sequence 905 and a capture domain (e.g., a poly-A sequence) 906, the first probe 901 and a second probe 904 hybridize 910 to an analyte 907. A ligase 921 ligates 920 the first probe to the second probe thereby generating a ligation product 922. The ligation product is released 930 from the analyte 931 by digesting the analyte using an endoribonuclease 932. The sample is permeabilized 940 and the ligation product 941 is able to hybridize to a capture probe on the substrate. Methods and composition for spatial detection using templated ligation have been described in PCT Publ. No. WO 2021/133849 A1, U.S. Pat. Nos. 11,332,790 and 11,505,828, each of which is incorporated by reference in its entirety.

In some embodiments, as shown in FIG. 9B, the ligation product 9001 includes a capture probe capture domain 9002, which can bind to a capture probe 9003 (e.g., a capture probe immobilized, directly or indirectly, on a substrate 9004). In some embodiments, methods provided herein include contacting 9005 a biological sample with a substrate 9004, wherein the capture probe 9003 is affixed to the substrate (e.g., immobilized to the substrate, directly or indirectly). In some embodiments, the capture probe capture domain 9002 of the ligation product specifically binds to the capture domain 9006. The capture probe can also include a unique molecular identifier (UMI) 9007, a spatial barcode 9008, a functional sequence 9009, and a cleavage domain 9010.

In some embodiments, methods provided herein include permeabilization of the biological sample such that the capture probe can more easily capture the ligation products (i.e., compared to no permeabilization). In some embodiments, reverse transcription (RT) reagents can be added to permeabilized biological samples. Incubation with the RT reagents can extend the capture probes 9011 to produce spatially-barcoded full-length cDNA 9012 and 9013 from the captured ligation products (e.g., ligation products).

In some embodiments, the extended ligation products can be denatured 9014 from the capture probe and transferred (e.g., to a clean tube) for downstream processing, such as, amplification, and/or library construction. The spatially-barcoded ligation products can be amplified 9015 via PCR prior to library construction. P5 9016 and P7 9019 can be used as sequences that are complementary to sequencing probes for immobilization of the library on the sequencing flow cell and i5 9017 and i7 9018 can be used as sample indexes. The resulting amplicons can then be sequenced using paired-end sequencing using TruSeq Read 1 and TruSeq Read 2 as sequencing primer sites.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) an analyte capture sequence. As used herein, the term “analyte binding moiety barcode” refers to a barcode that is associated with or otherwise identifies the analyte binding moiety. As used herein, the term “analyte capture sequence” refers to a region or moiety configured to hybridize to, bind to, couple to, or otherwise interact with a capture domain of a capture probe. In some cases, an analyte binding moiety barcode (or portion thereof) may be able to be removed (e.g., cleaved) from the analyte capture agent. Additional description of analyte capture agents can be found in Section (II)(b)(ix) of PCT Publication No. WO2020/176788 and/or Section (II)(b) (viii) U.S. Patent Application Publication No. 2020/0277663.

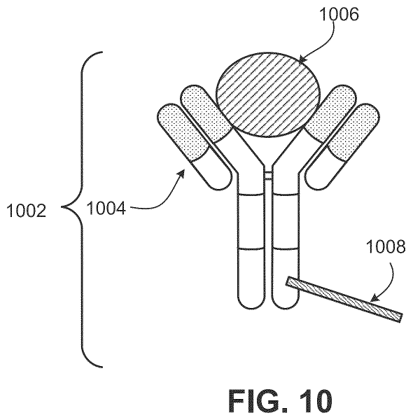

FIG. 10 is a schematic diagram of an exemplary analyte capture agent 1002 comprised of an analyte-binding moiety 1004 and an analyte-binding moiety barcode domain 1008. The exemplary analyte-binding moiety 1004 is a molecule capable of binding to an analyte 1006 and the analyte capture agent is capable of interacting with a spatially-barcoded capture probe. The analyte-binding moiety can bind to the analyte 1006 with high affinity and/or with high specificity. The analyte capture agent can include an analyte-binding moiety barcode domain 1008, a nucleotide sequence (e.g., an oligonucleotide), which can hybridize to at least a portion or an entirety of a capture domain of a capture probe. The analyte-binding moiety barcode domain 1008 can comprise an analyte binding moiety barcode and a capture handle sequence described herein. The analyte-binding moiety 1004 can include a polypeptide and/or an aptamer. The analyte-binding moiety 1004 can include an antibody or antibody fragment (e.g., an antigen-binding fragment).

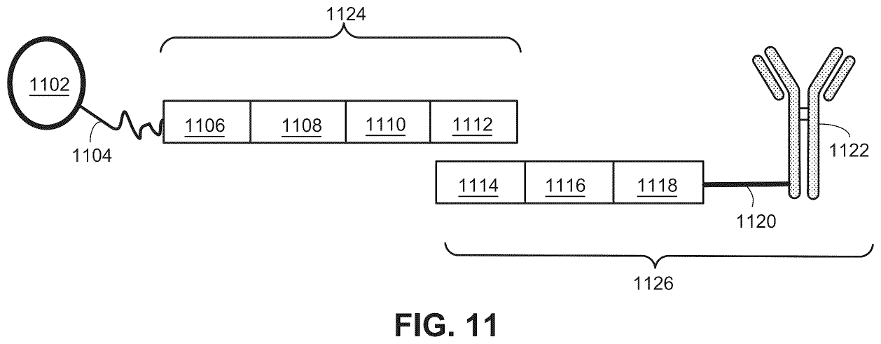

FIG. 11 is a schematic diagram depicting an exemplary interaction between a feature-immobilized capture probe 1124 and an analyte capture agent 1126. The feature-immobilized capture probe 1124 can include a spatial barcode 1108 as well as functional sequences 1106 and UMI 1110, as described elsewhere herein. The capture probe can be affixed 1104 to a feature (e.g., bead) or array 1102. The capture probe can also include a capture domain 1112 that is capable of binding to an analyte capture agent 1126. The analyte capture agent 1126 can include a functional sequence 1118, analyte binding moiety barcode 1116, and a capture handle sequence 1114 that is capable of binding to the capture domain 1112 of the capture probe 1124. The analyte capture agent can also include a linker 1120 that allows the capture agent barcode domain 1116 to couple to the analyte binding moiety 1122.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of PCT Publication No. WO2020/176788 and/or U.S. Patent Application Publication No. 2020/0277663. See, for example, the Exemplary embodiment starting with “In some non-limiting examples of the workflows described herein, the sample can be immersed . . . ” of PCT Publication No. WO2020/176788 and/or U.S. Patent Application Publication No. 2020/0277663. See also, e.g., the Visium Spatial Gene Expression Reagent Kits User Guide (e.g., Rev F, dated January 2022); and/or the Visium Spatial Gene Expression Reagent Kits-Tissue Optimization User Guide (e.g., Rev E, dated February 2022).

In some embodiments, spatial analysis can be performed using dedicated hardware and/or software, such as any of the systems described in Sections (II)(e)(ii) and/or (V) of PCT Publication No. WO2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of PCT Publication No. WO2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Publication No. WO2021/102003 and/or U.S. Patent Application Publication No. 2021/0150707, each of which is incorporated herein by reference in their entireties.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Publication No. WO2020/053655 and spatial analysis methods are generally described in PCT Publication No. WO2021/102039 and/or U.S. Patent Application Publication No. 2021/0155982, each of which is incorporated herein by reference in their entireties.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of PCT Publication Nos. WO2020/123320, WO 2021/102005, and/or U.S. Patent Application Publication No. 2021/0158522, each of which is incorporated herein by reference in their entireties. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

### B. Mitigation of “Hot Edge” Effect of Mislocalization of Analytes

Provided herein are methods for reducing mislocalization of target nucleic acids from a biological sample, the method including: (a) providing an array, wherein the array includes a first region of capture probes and a second region of capture probes, where capture probes of the first region of capture probes include: (i) a spatial barcode, (ii) a first capture domain, and (iii) one or more functional domains including a primer binding site or a sequencing specific site, and capture probes of the second region of capture probes include a second capture domain; and (b) hybridizing target nucleic acids from the biological sample to the first capture domain of capture probes of the first region and a second capture domain of the capture probes of the second region, wherein the capture probes of the second region do not comprise one or more of a primer binding site or sequencing specific site (i.e., the capture probes in the second region do not include the primer binding site or sequencing specific site as the capture probes in the first region), such that hybridization of the target nucleic acids to the capture probes of the second region of capture probes reduces mislocalization of target nucleic acids.

Also provided herein are methods of reducing sequencing output from mislocalized target nucleic acids from a biological sample, the methods including: (a) providing an array, wherein the array includes a first region of capture probes and a second region of capture probes, wherein capture probes of the first region include: (i) a spatial barcode, (ii) a first capture domain, and (iii) one or more functional domains, and capture probes of the second region include a second capture domain; (b) contacting target nucleic acids from the biological sample with the plurality of capture probes on the array wherein the capture probes of the first and second regions hybridize to the target nucleic acids in the first and second regions proximal to their location in the biological sample; (c) extending the capture probes hybridized to the target nucleic acids, thereby generating extension products, and (d) determining the sequence of the (i) spatial barcode or a complement thereof and (ii) a portion of the target nucleic acid or a complement thereof from the extension products from capture probes in the first region, thereby reducing sequencing output for mislocalized target nucleic acids from the biological sample.

Also provided herein are methods for reducing mislocalization of target nucleic acids from a biological sample, the method including: (a) providing an array, where the array includes a first region of capture probes and a second region of capture probes, where capture probes of the first region of capture probes include: (i) a spatial barcode, (ii) a capture domain, and (iii) one or more functional domains, and capture probes of the second region of capture probes comprise a capture domain; and (b) hybridizing target nucleic acids from the biological sample to the capture domains of capture probes of the first region and the capture probes of the second region, such that hybridization of the target nucleic acids to the capture probes of the second region of capture probes reduces mislocalization of target nucleic acids.

As described above, array-based spatial transcriptomics methods can measure analyte and/or gene expression data for a variety of analytes (e.g., target nucleic acids), or proxies thereof (e.g., ligation products, analyte binding moiety barcodes, etc.), within a biological sample at high spatial resolution, while retaining native spatial context.

In some embodiments, a ligation product is produced by hybridizing a first probe and a second probe to an analyte (e.g., a nucleic acid analyte). In some embodiments, the biological sample is contacted with about 5,000 or more probe pairs collectively including the first probe and the second probe. In some embodiments, the first probe and/or the second probe is a DNA probe. In some embodiments, the first probe and/or the second probe include a primer sequence (e.g., a sequence that does not hybridize to the analyte). In some embodiments, the first probe or the second probe includes a capture probe binding domain. The capture probe binding domain is a sequence substantially complementary to the capture domain of a capture probe on the array. In some embodiments, the capture probe binding domain hybridizes to the capture domain of the capture probe.

In some embodiments, the first probe and the second probe are substantially complementary to adjacent sequences of the nucleic acid analyte (e.g., the first probe and second probe abut one another as they are hybridized to the analyte). As used herein “adjacent” means there are no intervening nucleotides between the first probe and the second probe. In some embodiments, the first probe and the second probe hybridize to sequences that are not adjacent to each other on the analyte (e.g., 2, 3, 4, 5, 6, 7, 8, 9, 10, 11, 12, 13, 14, 15, or more intervening nucleotides). In some embodiments, the first probe is extended with a DNA polymerase, thereby (i) filling in a gap between the first probe and the second probe and (ii) generating an extended first probe. In such embodiments, the extended first probe is ligated to the second probe.

In some embodiments, coupling includes ligating the first probe and the second probe via a ligase. In some embodiments, the ligase is one or more of a T4 RNA ligase (Rnl2), a PBCV-1 DNA ligase, a Chlorella virus DNA ligase, a single-stranded DNA ligase, a T4 DNA ligase, and combinations thereof.

In some embodiments, the analyte is a nucleic acid. In some embodiments, the nucleic acid is an RNA molecule. In some embodiments, the RNA molecule is mRNA. In some embodiments, the nucleic acid is a DNA molecule. In some embodiments, the DNA molecule is genomic DNA.

In some embodiments, the systems for performing spatial analysis include components such as a chamber (e.g., a flow cell or sealable, fluid-tight chamber) for containing a biological sample. The biological sample can be mounted for example, in a biological sample holder. In some embodiments, the biological sample is disposed on a first substrate and aligned with an array disposed on a second substrate, forming a chamber. This chamber configuration can facilitate diffusion and/or migration of analytes (e.g., target nucleic acids), or proxies thereof (e.g., ligation products, analyte capture sequences) from the biological sample to the array. Subsequent analysis of the transferred analytes or proxies thereof includes determining the identity of the analytes or proxies thereof and the spatial location of the analytes within the biological sample. The spatial location of an analyte within the biological sample is determined based on the spatial barcode of the capture probe to which the analyte or proxy thereof is bound (e.g., directly or indirectly) on the array, and the capture probe's relative spatial location within the array. Therefore, accurate measurement of the location and/or abundance of an analyte within the biological sample depends on faithful localization of the analyte to the capture probe with which it is aligned on the array.

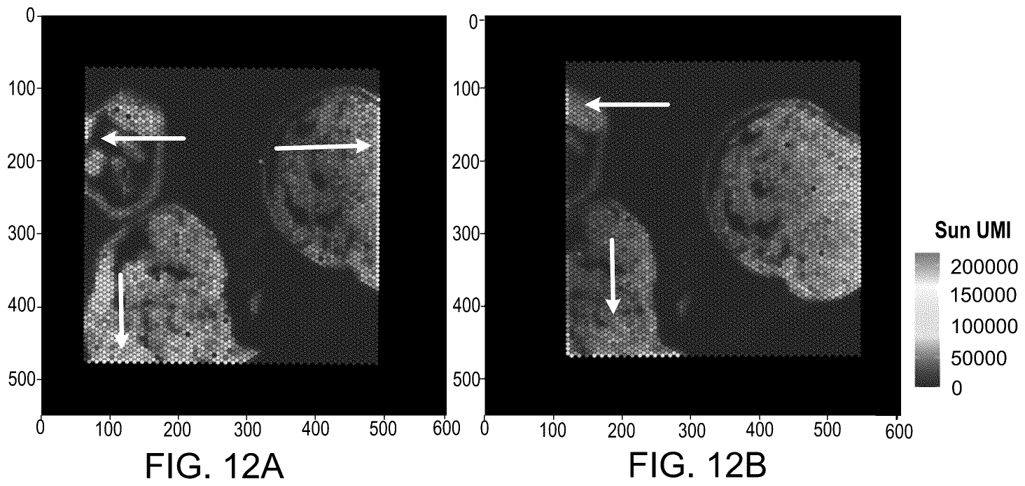

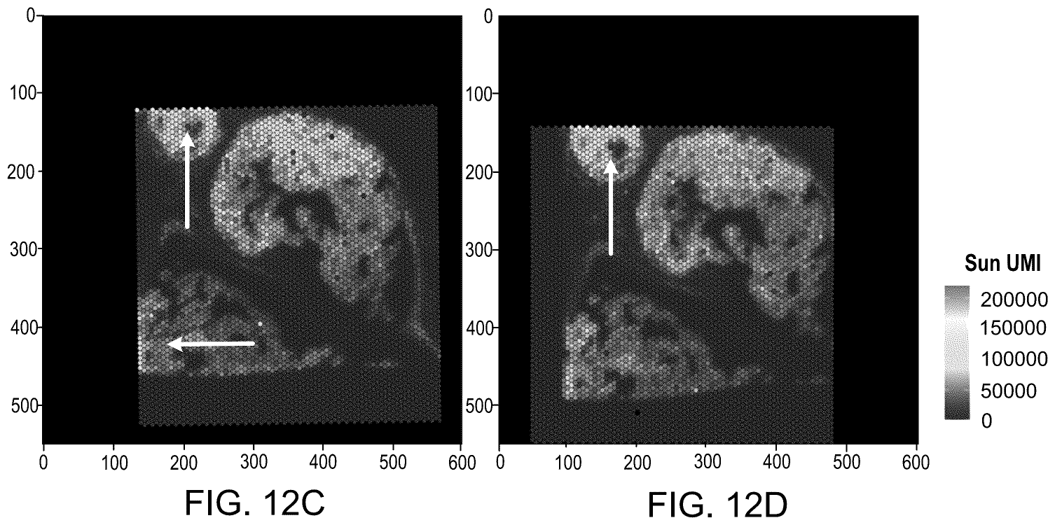

In some cases, it is possible that the biological sample exceeds the boundaries of the array resulting in one or more areas of the biological sample that do not have capture probes underneath or are not aligned to capture probes. This can result in mislocalization of analytes (e.g., target nucleic acids) from cells of the biological sample that were not aligned with features of the array to the most proximate features on the array. For example, during spatial analysis including capture probes on an array, the edges or boundaries of the array can bind analytes or proxies thereof from tissue that exceeded the boundaries of the capture probes on the array, leading to a “hot edge” scenario were the capture probes along the perimeter or the boundary of the array capture target analytes or proxies thereof from the biological sample that are not aligned over the array, resulting in artificially elevated detection and subsequent data from those features. This hot edge signal pattern can distort the sequencing data and reduce the accuracy of the spatial analysis. FIGS. 12A-12D each show exemplary UMI count readouts of an array-based spatial transcriptomics assay illustrating the hot edge signal pattern. In each image, arrows point to probes at the periphery of the array that represent artificially elevated detection as a result of target analytes from a biological sample migrating to those boundary features due to mislocalization from an area of no capture probes.

While not wishing to be bound by theory, it is thought that during transfer of analytes (e.g., target nucleic acids) from the biological sample and capture of the analytes by the capture probes, analytes migrating from cells that are aligned with capture probes get captured and effectively reach the binding capacity of the capture probes at that feature due to the proximity of the capture probes to the target analytes from the biological sample. In some embodiments, the capture probes on the array are in excess of the analytes migrating from the biological sample and therefore there is an excess of capture probes at the feature relative to the analytes that migrate from the cells of the biological sample superior to the arrayed area. It is contemplated that analytes migrating from cells that are not aligned with capture probes are free to diffuse and can mislocalize and hybridize to adjacent capture probes that are not in proximity to the location of the analyte in the biological sample, resulting in the hot edge signal pattern on the outmost capture probes at the periphery of the array where the biological sample extends beyond the boundaries of the capture probes on the array.

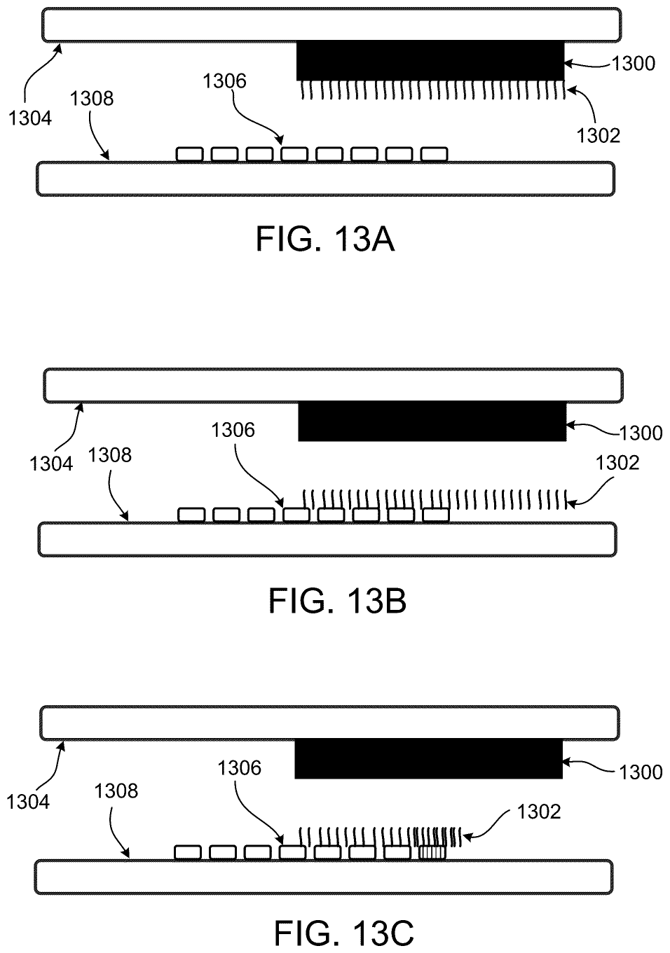

FIGS. 13A-13C illustrate the proposed mechanisms leading to the hot edge signal pattern. FIGS. 13A-13C depict an exemplary cross-sectional view of a spatial array platform. As shown in FIG. 13A, a biological sample 1300 is disposed on a substrate 1304. Features 1306 are arrayed on substrate 1308. Features 1306 can include capture probes embedded, spotted, printed, fabricated, etc. on the substrate 1308, or a substrate 1308 with features 1306 (e.g., spots, beads, wells, posts, divots, pegs, etc.) comprising capture probes. The biological sample 1300 includes analytes or proxies thereof 1302. Notably, a portion of the biological sample 1300 is aligned with features 1306 and a portion of the biological sample 1300 is not aligned with features comprising capture probes 1306, i.e., a portion of the biological sample exceeds the boundaries of the array features 1306 on substrate 1308.

As shown in FIG. 13B, in an exemplary embodiment, analytes 1302 have migrated from the biological sample 1300 to the substrate 1308. Analytes migrating from the portion of the biological sample 1300 that is aligned with features 1306 are captured by capture probes 1306 inferior/proximal to their location in the biological sample. Analytes migrating from the portion of the biological sample 1300 that is not aligned with features 1306 are not immediately captured by capture probes of a feature, as there are no features inferior to that portion of the biological sample.

As shown in FIG. 13C, the analytes 1302 that migrated from the portion of the biological sample 1300 that is not aligned with features 1306 comprising capture probes, and were therefore not captured, are free to diffuse through the reaction fluid to adjacent features 1306 on the periphery of the array, thereby mislocalizing and hybridizing to probes that are not inferior to their location in a biological sample. For example, in FIG. 13C, the grey shaded feature has captured a portion of the analytes 1302 that migrated from the portion of the biological sample 1300 that is not aligned with features 1306. Therefore, the capture probes of the grey shaded feature in have captured analytes from the biological sample superior to the feature as well as mislocalized analytes from the biological sample not directly above an arrayed feature. When sequenced, the analytes collected from the grey shaded feature will read out artificially elevated UMI counts and additional sequencing data, thereby distorting the measurement of the location and abundance of analytes in the biological sample.

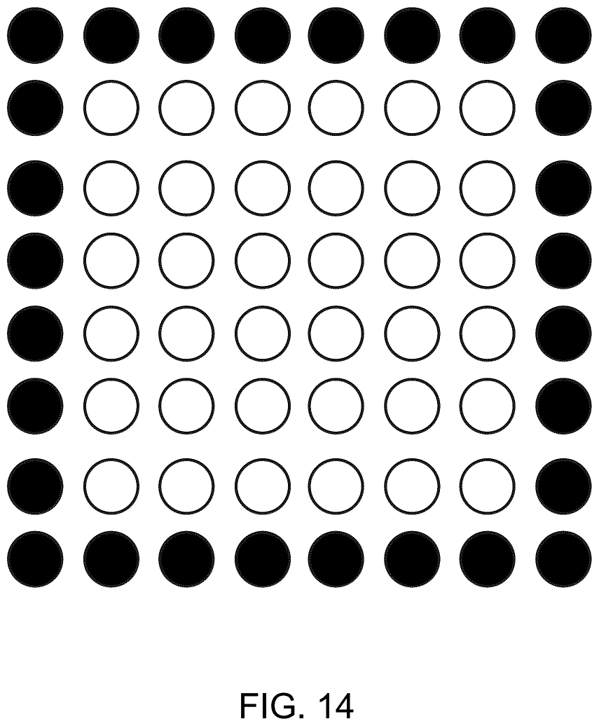

As shown in FIG. 14, an array of features can be fabricated to mitigate the hot edge signal pattern. Embodiments of the array-based spatial transcriptomics assay include the use of a capture probe comprising a spatial barcode for analyte spatial correlation in a biological sample and a capture domain that is capable of hybridizing to a nucleic acid or a proxy thereof produced by and/or present in a cell of the biological sample. Generally, analytes and/or proxies thereof can be captured when contacting a biological sample with a substrate including capture probes (e.g., a substrate with capture probes embedded, spotted, printed, fabricated on the substrate, or a substrate with features (e.g., beads, wells, spots, etc.) comprising capture probes). In some embodiments, in order to mitigate the hot edge signal pattern, capture probes at the interior of the array, representing a majority of the capture probes and depicted in white in FIG. 14, include a capture domain that is capable of hybridizing to an analyte or proxy thereof, a spatial barcode, and one or more of a unique molecular identifier (UMIs), a cleavage domain, and/or one or more functional domains. Capture probes on the boundaries or around the perimeter of the array, depicted in black in FIG. 14, can include a capture domain that is capable of hybridizing an analyte or a proxy thereof, but preferably do not include a primer binding site (e.g., a sequencing or primer binding site) and/or amplification domain. In some embodiments, capture probes on the boundaries or around the perimeter of the array further do not include a spatial barcode, or additional domains. Therefore, capture probes at the boundaries of the array, depicted in black in FIG. 14, capture analytes or proxies thereof, but nucleic acid products generated from these capture probes are not subjected to further processing steps or downstream analysis such as sequencing.

Referring again to FIG. 14, in some embodiments, capture probes at the interior of the array depicted in white may be configured to prime, replicate, and consequently yield barcoded extension products from a template (e.g., a DNA or RNA template (e.g., mRNA), such as an analyte or a proxy thereof (e.g., a ligation product or an analyte capture agent), or a portion thereof), or derivatives thereof. In some embodiments, extended capture probes are amplified (e.g., in bulk solution or on the array) to yield quantities that are sufficient for downstream analysis, e.g., sequencing. In some embodiments, extended capture probes (e.g., DNA molecules) can act as templates for an amplification reaction (e.g., a polymerase chain reaction). In some embodiments, a second strand complementary to the extended capture probe is generated. Capture probes at the boundaries of the array are not amplified due to not having a functional domain, (e.g., a primer binding site) and are further not analyzed downstream by, e.g., sequencing (e.g., no sequencing specific domain).

In some embodiments, the capture probes at the interior of the array, which can include a capture domain that is capable of binding to an analyte (e.g., target nucleic acids) or a proxy thereof and can further include a spatial barcode, unique molecular identifiers (UMIs), and/or functional domains form a first region of capture probes, and the capture probes at the boundaries or periphery of the array form a second region of capture probes. In some embodiments, the second region of capture probes surrounds, or forms a perimeter around, the first region of capture probes. In some embodiments where the capture probes are affixed to features arrayed in rows and columns on a substrate, the second region of capture probes comprises 1, 2, 3, 4, 5, 6, 7, 8, 9, 10 rows or more of features at the perimeter of the array and/or 1, 2, 3, 4, 5, 6, 7, 8, 9, 10 columns or more of features at the perimeter of the array. In some embodiments, the second region of capture probes comprises a lawn of capture probes forming a perimeter surrounding the first region of capture probes on the array. In some embodiments, the second region of capture probes comprises a continuous coating of capture probes forming a perimeter surrounding the first region of capture probes on the array. In some embodiments, the density of capture probes is higher in the second region of capture probes than in the first region of capture probes. In some embodiments, the perimeter of the array includes a peripheral edge of a plurality of patterned features on the spatial array. In some embodiments, the second region of capture probes is arrayed in a pattern that is different than the first region of capture probes, e.g., the second region of capture probes can be arrayed on the substrate in a hexagonal pattern while the first region of capture probes is arrayed in a square grid pattern on the substrate.

As discussed above, the capture probes include a capture domain. In some embodiments, the capture probes of the second region of capture probes specifically hybridize to analytes (e.g., target nucleic acids) or proxies thereof, e.g., analytes or their proxies that have migrated from the portion of the biological sample is not aligned with the first region of capture probes. In some embodiments, the capture probes of the second region of capture probes non-specifically hybridize to analytes, e.g., analytes that have migrated from the portion of the biological sample is not aligned with the first region of capture probes. In some embodiments, the capture domains of the capture probes are the same in the first region (e.g., first capture domains) and the second region (e.g., second capture domains) and include a homopolymer sequence, such as a poly(T) sequence. In some embodiments, the capture domains of the capture probes are the same in the first region (e.g., first capture domains) and the second region (e.g., second capture domains) and include a non-homopolymeric sequence. In some embodiments, the capture domains of the capture probes are different in the first region (e.g., first capture domains) and the second region of capture probes (e.g., second capture domains).

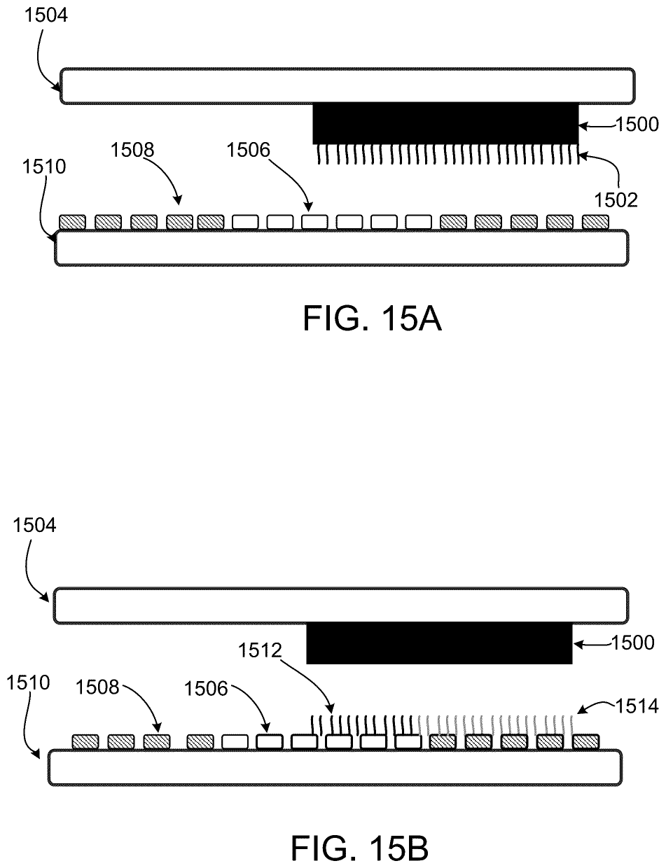

FIGS. 15A-15B illustrate compositions and methods for mitigating the hot edge signal pattern. FIGS. 15A-15B depicts a cross-sectional view of a spatial array platform. As shown in FIG. 15A, a biological sample 1500 is disposed on a substrate 1504. A first region of features 1506 and a second region of features 1508 are arrayed on substrate 1510. The second region of features 1508 surrounds and forms a perimeter around the first region of features 1506. Features 1506 can include capture probes embedded, spotted, printed, fabricated on the substrate 1510, wherein the capture probes include a capture domain that is capable of binding to an analyte, or proxy thereof, and further include a spatial barcode, a unique molecular identifier (UMI), and/or functional domains. Features 1508 can include capture probes embedded, spotted, printed, fabricated on the substrate 1510, wherein the capture probes include a capture domain that is capable of binding to an analyte or a proxy thereof, but do not include a sequencing primer binding site and/or amplification domain and optionally, a spatial barcode, a unique molecular identifier (UMI), and/or functional domains. The biological sample 1500 includes analytes 1502 (or proxies thereof). Notably, a portion of the biological sample 1500 is aligned with the first region of features 1506 and a portion of the biological sample 1500 is aligned with the second region of features 1508.

As shown in FIG. 15B, analytes or proxies thereof 1502 have migrated from the biological sample 1500 to the substrate 1510. A portion of the analytes 1512 have been captured by capture probes in the first region of features 1506 and a portion of the analytes 1514 have been captured by capture probes in the second region of features 1508. Because the features 1506 include capture probes comprising a capture domain that is capable of binding to an analyte, or a proxy thereof, and further include a spatial barcode, unique molecular identifiers (UMIs), and/or functional domains, analytes 1512 captured at features 1506 will undergo downstream analysis, e.g., sequencing, and will produce detectable qualitative and/or quantitative spatial transcriptomics data. Because the features 1508 include capture probes comprising a capture domain that is capable of binding to an analyte, or a proxy thereof, and do not include a sequencing primer binding site and/or amplification domain (and optionally a spatial barcode, unique molecular identifier (UMIs), and/or functional domains), analytes 1514 captured at features 1508 will not undergo downstream analysis, e.g., sequencing, and will not produce detectable qualitative and/or quantitative signal and spatial transcriptomics data. The capture of analytes 1514 at the second region of features 1508 mitigates mislocalization of analytes 1514 captured at the first region of features 1506 thereby relieving a majority, if not all, of the hot edge signal pattern and improving the accuracy of the spatial transcriptomics assay.

In some embodiments, the method includes generating one or more extension products. For example, in embodiments where target nucleic acids are captured directly by capture probes (e.g., capture probes of the first region and/or the second region) the capture probe can be extended using the target nucleic acid as a template, thereby generating an extension product (i.e., an extended capture probe). Optionally, a complementary sequence (e.g., a second strand) to the extension product (i.e., an extended capture probe) can be generated. The generated complementary sequence incorporates a complementary sequence of the spatial barcode.

Similarly, in embodiments where ligation products are captured by capture probes (e.g., capture probes of the first region and/or the second region) the capture probe can be extended using the ligation product as a template, thereby generating an extension product (i.e., an extended capture probe). Optionally, a complementary sequence can be generated by extending the ligation product using the capture probe as a template, thereby generating an extended ligation product which incorporates a complementary sequence of the spatial barcode.

In some embodiments, the method includes generating a nucleic acid library for sequencing including extension products generated from target nucleic acids, or complements thereof (e.g., extended capture probes and their complementary sequences (e.g., extended target nucleic acids or second strand sequences)). In some embodiments, the method includes generating a nucleic acid library for sequencing including extension products generated from ligation products, or complements thereof (e.g., extended capture probes, extended ligation products).

In some embodiments, the method includes determining (i) the sequence of the spatial barcode or a complement thereof, and (ii) all or a portion of the sequence of the target nucleic acid, or a complement thereof, and using the determined sequences of (i) and (ii) to determine the locations of the target nucleic acids in the biological sample.

In some embodiments, the method includes determining (i) the sequence of the spatial barcode or a complement thereof, and (ii) all or a portion of the sequence of the ligation product, or a complement thereof, and using the determined sequences of (i) and (ii) to determine the locations of the target nucleic acids in the biological sample.

In some embodiments, the method includes amplifying any of the extension products described herein.

In some embodiments, the determining step includes sequencing. In some embodiments, the sequencing includes high-throughput sequencing. In some embodiments, the method includes sequencing the nucleic acid library with a sequencing primer that hybridizes to the sequencing specific site of capture probes in the first region.

In some embodiments, the method includes imaging the biological sample. In some embodiments, the method includes staining the biological sample. In some embodiments, the staining includes hematoxylin and/or eosin staining. In some embodiments, the staining includes the use of a detectable label selected from the group consisting of a radioisotope, a fluorophore, a chemiluminescent compound, a bioluminescent compound, or a combination thereof.

Biological samples can be stained using a wide variety of stains and staining techniques. In some embodiments, a biological sample can be stained using any number of biological stains, including but not limited to, acridine orange, Bismarck brown, carmine, coomassie blue, cresyl violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, or safranin.

The sample can be stained using known staining techniques, including Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS) staining techniques. PAS staining is typically performed after formalin or acetone fixation.

In some embodiments, staining includes identifying analytes using fluorescently-conjugated antibodies. In some embodiments, a biological sample is stained using two or more different types of stains, or two or more different staining techniques. For example, a biological sample can be prepared by staining and imaging using one technique (e.g., H&E staining and brightfield imaging), followed by staining and imaging using another technique (e.g., IHC/IF staining and fluorescence microscopy) for the same biological sample. In some embodiments, immunofluorescence or immunohistochemistry protocols (direct and indirect staining techniques) can be performed as a part of, or in addition to, the exemplary spatial workflows presented herein.

In some embodiments, the biological sample is permeabilized. In some embodiments, the permeabilizing includes the use of an organic solvent, a detergent, and an enzyme, or a combination thereof. In some embodiments, permeabilizing includes the use of an endopeptidase, a protease, sodium dodecyl sulfate, polyethylene glycol tert-octylphenyl ether, polysorbate 80, polysorbate 20, N-lauroylsarcosine sodium salt solution, saponin, Triton X-100™, Tween-20™, or combinations thereof. In some embodiments, permeabilizing includes and endopeptidase, such as pepsin, or proteinase K. In some embodiments, the biological sample is permeabilized prior to hybridizing the first probe and the second probe to the analyte.

In some embodiments, the array includes one or more features selected from the group consisting of: a spot, an inkjet spot, a masked spot, a pit, a post, a well, a ridge, a divot, a hydrogel pad, and a bead. For example, in some embodiments, the array includes a gel containing capture probes. In some embodiments, the array includes a plurality of beads each comprising a plurality of capture probes.

In some embodiments, the determining step comprises sequencing. In some embodiments, the sequencing is high-throughput sequencing.

In some embodiments, the biological sample is a tissue sample. In some embodiments, the tissue sample is a fresh-frozen tissue sample. In some embodiments, the tissue sample is a fixed tissue sample, and optionally, where the fixed tissue sample is a formalin-fixed paraffin-embedded tissue sample, an acetone-fixed tissue sample, a methanol-fixed tissue sample, or a paraformaldehyde-fixed tissue sample. In some embodiments, the biological sample is a tissue section. In some embodiments, the tissue section is a fresh-frozen tissue section. In some embodiments, the tissue section is a fixed tissue section. In some embodiments, the fixed tissue section is a formalin-fixed paraffin-embedded tissue section, an acetone-fixed tissue section, a methanol-fixed tissue section, or a paraformaldehyde-fixed tissue section.

### C. Sandwich Processes

In some embodiments, analytes or proxies thereof from the biological sample are released from the biological sample and captured by any of the methods described herein, for example, by capture probes of the first and second region of capture probes depicted in FIG. 14. In some embodiments, the biological sample is disposed directly on the array of capture probes. In some embodiments, the biological sample is mounted on a first substrate and the substrate comprising the array comprising the first and second region of capture probes is a second substrate. In such embodiments, alignment of the first substrate and the second substrate can be facilitated by a sandwiching process. Accordingly, described herein are methods, compositions, and kits for sandwiching together the first substrate as described herein with a second substrate having an array with the first and second region of capture probes.

In some embodiments, the sandwiching process may be facilitated by a device, sample holder, sample handling apparatus, or system described in, e.g., US. Patent Application Pub. No. 20210189475, PCT Publ. No. WO 2021/252747, or PCT Publ. No. WO 2022/061152.

In some embodiments, the first and second substrates are placed in a substrate holder (e.g., an array alignment device) configured to align the biological sample and the array comprising the first and second region of capture probes. In some embodiments, the device comprises a sample holder. In some embodiments, the sample holder includes a first member and a second member that receive a first substrate and a second substrate, respectively. The device can include an alignment mechanism that is connected to at least one of the members and aligns the first and second members. Thus, the devices of the disclosure can advantageously align the first substrate and the second substrate and any samples, the first and second region of capture probes, or permeabilization reagents that may be on the surface of the first and second substrates.

In some embodiments, the sandwiching process comprises: mounting the first substrate on a first member of a support device, the first member configured to retain the first substrate; mounting the second substrate on a second member of the support device, the second member configured to retain the second substrate, applying a reagent medium to the first substrate and/or the second substrate, the reagent medium comprising a permeabilization agent, operating an alignment mechanism of the support device to move the first member and/or the second member such that a portion of the biological sample is aligned (e.g., vertically aligned) with a portion of the array including the first and second region of capture probes and within a threshold distance of the first and second region of capture probes, and such that the portion of the biological sample and the capture probe contact the reagent medium, wherein the permeabilization agent releases the analytes or proxies thereof from the biological sample.

In some embodiments of a sample holder, the sample holder can include a first member including a first retaining mechanism configured to retain a first substrate comprising a sample. The first retaining mechanism can be configured to retain the first substrate disposed in a first plane. The sample holder can further include a second member including a second retaining mechanism configured to retain a second substrate disposed in a second plane. The sample holder can further include an alignment mechanism connected to one or both of the first member and the second member. The alignment mechanism can be configured to align the first and second members along the first plane and/or the second plane such that the sample contacts at least a portion of the reagent medium when the first and second members are aligned and within a threshold distance along an axis orthogonal to the second plane. The alignment mechanism may be configured to move the second member along the axis orthogonal to the second plane and/or move the first member along an axis orthogonal to the first plane.

In some embodiments, the alignment mechanism includes a linear actuator. In some embodiments, the alignment mechanism includes one or more of a moving plate, a bushing, a shoulder screw, a motor bracket, and a linear actuator. The moving plate may be coupled to the first member or the second member. The alignment mechanism may, in some cases, include a first moving plate coupled to the first member and a second moving plate coupled to the second member. In some embodiments, the linear actuator is configured to move the second member along an axis orthogonal to the plane of the first member and/or the second member. For example, the moving plate may be coupled to the second member and adjust the separation distance along a z axis (e.g., orthogonal to the second substrate) by moving the moving plate up in a superior direction toward the first substrate. In some embodiments, the linear actuator is configured to move the first member along an axis orthogonal to the plane of the first member and/or the second member. The movement of the moving plate may be accomplished by the linear actuator configured to move the first member and/or the second member at a velocity. The velocity may be controlled by a controller communicatively coupled to the linear actuator. In some embodiments, the linear actuator is configured to move the first member, the second member, or both the first member and the second member at a velocity of at least 0.1 mm/sec (e.g., at least 0.1 mm/sec to 2 mm/sec). In some aspects, the velocity may be selected to reduce or minimize bubble generation or trapping within the reagent medium. In some embodiments, the linear actuator is configured to move the first member, the second member, or both the first member and the second member with an amount of force of at least 0.1 lbs (e.g., between 0.1-4.0 pounds of force).

In some aspects, the velocity of the moving plate (e.g., closing the sandwich) may affect bubble generation or trapping within the reagent medium. It may be advantageous to minimize bubble generation or trapping within the reagent medium during the “sandwiching” process, as bubbles can interfere with the migration of analytes or proxies thereof through the reagent medium to the array. In some embodiments, the closing speed is selected to minimize bubble generation or trapping within the reagent medium. In some embodiments, the closing speed is selected to reduce the time it takes the flow front of the reagent medium from an initial point of contact with the first and second substrate to sweep across the sandwich area (also referred to herein as “closing time”). In some embodiments, the closing speed is selected to reduce the closing time to less than about 1100 milliseconds (ms). In some embodiments, the closing speed is selected to reduce the closing time to less than about 1000 ms. In some embodiments, the closing speed is selected to reduce the closing time to less than about 900 ms. In some embodiments, the closing speed is selected to reduce the closing time to less than about 750 ms. In some embodiments, the closing speed is selected to reduce the closing time to less than about 600 ms. In some embodiments, the closing speed is selected to reduce the closing time to about 550 ms or less. In some embodiments, the closing speed is selected to reduce the closing time to about 370 ms or less. In some embodiments, the closing speed is selected to reduce the closing time to about 200 ms or less. In some embodiments, the closing speed is selected to reduce the closing time to about 150 ms or less.

Further details on support devices, sample holders, sample handling apparatuses, or systems for implementing a sandwiching process are described in, e.g., PCT Publ. No. WO 2021/0189475 and PCT Publ. No. WO 2022/061152, each of which are incorporated by reference in their entireties.

Analytes or proxies thereof within a biological sample may be released through disruption (e.g., permeabilization, digestion, etc.) of the biological sample or may be released without disruption. Various methods of permeabilizing (e.g., any of the permeabilization reagents and/or conditions described herein) a biological sample are described herein, including for example including the use of various detergents, buffers, proteases, and/or nucleases for different periods of time and at various temperatures. Additionally, various methods of delivering fluids (e.g., a buffer, a permeabilization solution) to a biological sample are described herein including the use of a substrate holder (e.g., for sandwich assembly, sandwich configuration, as described herein)

Provided herein are methods for delivering a fluid to a biological sample disposed on an area of a first substrate and an array comprising the first and second region of capture probes disposed on a second substrate.

In some embodiments, the sandwich configuration described herein between a first substrate comprising a biological sample and a second substrate comprising array comprising the first and second region of capture probes may include a reagent medium comprising NaCl, ethylene carbonate, and/or glycerol to fill a gap. It may be desirable that the reagent medium be free from air bubbles between the slides to facilitate transfer of target molecules with spatial information. Additionally, air bubbles present between the slides may obscure at least a portion of an image capture of a desired region of interest. Accordingly, it may be desirable to ensure or encourage suppression and/or elimination of air bubbles between the two substrates during a permeabilization step.

In some aspects, it may be possible to reduce or eliminate bubble formation between the slides using a variety of filling methods and/or closing methods.

In some embodiments, the contacting comprises bringing the two substrates into proximity such that the sample on the first substrate is aligned with the first and second region of capture probes on the second substrate.

In some embodiments, the drop includes permeabilization reagents (e.g., any of the permeabilization reagents described herein). In some embodiments, the rate of permeabilization of the biological sample is modulated by delivering the permeabilization reagents (e.g., a fluid containing permeabilization reagents) at various temperatures.

Robust fluidics in the sandwich making described herein may preserve spatial information by reducing or preventing deflection of molecules as they move from the tissue slide to the capture slide.

Further details on angled closure workflows, and devices and systems for implementing an angled closure workflow, are described in PCT Publ. No. WO 2021/252747 and PCT Publ. No. WO 2022/061152, which are hereby incorporated by reference in their entireties. Additional configurations for reducing or eliminating bubble formation, and/or for reducing unwanted fluid flow, are described in PCT Publ. No. WO 2021/252747, which is hereby incorporated by reference in its entirety.

Suitable permeabilization agents include, but are not limited to, organic solvents (e.g., acetone, ethanol, and methanol), cross-linking agents (e.g., paraformaldehyde), detergents (e.g., saponin, Triton X-100™, Tween-20™, or sodium dodecyl sulfate (SDS)), and enzymes (e.g., trypsin, proteases (e.g., proteinase K). In some embodiments, the detergent is an anionic detergent (e.g., SDS or N-lauroylsarcosine sodium salt solution). Exemplary permeabilization reagents are described in in PCT Patent Application Publication No. WO 2020/123320, which is incorporated by reference in its entirety.

Lysis solutions can include ionic surfactants such as, for example, sarkosyl and sodium dodecyl sulfate (SDS). More generally, chemical lysis agents can include, without limitation, organic solvents, chelating agents, detergents, surfactants, and chaotropic agents. Exemplary lysis reagents are described in PCT Patent Application Publication No. WO 2020/123320, which is incorporated by reference in its entirety.

Exemplary proteases include, e.g., pepsin, trypsin, pepsin, elastase, collagenase, and proteinase K. Exemplary proteases are described in PCT Patent Application Publication No. WO 2020/123320, which is incorporated by reference in its entirety. Exemplary detergents include sodium dodecyl sulfate (SDS), sarkosyl, saponin, Triton X-100™, and Tween-20™ Exemplary detergents are described in PCT Patent Application Publication No. WO 2020/123320, which is incorporated by reference in its entirety.

In some embodiments, the reagent medium also comprises a nuclease. In some embodiments, the nuclease comprises am RNase. In some embodiments, the RNase is selected from RNase A, RNase C, RNase H, and RNase I. In some embodiments, the reagent medium comprises one or more of sodium dodecyl sulfate (SDS), proteinase K, pepsin, N-lauroylsarcosine, RNase, and a sodium salt thereof.

The sample holder is compatible with a variety of different schemes for contacting the aligned portions of the biological sample and reusable array with the reagent medium to promote capture of the analytes or proxies thereof. In some embodiments, the reagent medium is deposited directly on the second substrate (e.g., forming a reagent medium that includes the permeabilization reagent and the feature array), and/or directly on the first substrate. In some embodiments, the reagent medium is deposited on the first and/or second substrate, and then the first and second substrates aligned in the sandwich configuration such that the reagent medium contacts the aligned portions of the biological sample and array. In some embodiments, the reagent medium is introduced into the gap while the first and second substrates are aligned in the sandwich configuration.

In certain embodiments a dried permeabilization reagent is applied or formed as a layer on the first substrate or the second substrate or both prior to contacting the sample and the feature array. For example, a reagent can be deposited in solution on the first substrate or the second substrate or both and then dried. Drying methods include but are not limited to spin coating a thin solution of the reagent and then evaporating a solvent included in the reagent or the reagent itself. Alternatively, in other embodiments, the reagent can be applied in dried form directly onto the first substrate or the second substrate or both. In some embodiments, the coating process can be done in advance of the analytical workflow and the first substrate, and the second substrate can be stored pre-coated. Alternatively, the coating process can be done as part of the analytical workflow. In some embodiments, the reagent is a permeabilization reagent. In some embodiments, the reagent is a permeabilization enzyme, a buffer, a detergent, or any combination thereof. In some embodiments, the permeabilization enzyme is pepsin. In some embodiments, the reagent is a dried reagent (e.g., a reagent free from moisture or liquid). In some instances, the substrate that includes the sample (e.g., a histological tissue section) is hydrated. The sample can be hydrated by contacting the sample with a reagent medium, e.g., a buffer that does not include a permeabilization reagent. In some embodiments, the hydration is performed while the first and second substrates are aligned in a sandwich configuration.

In some embodiments, following initial contact between sample and a permeabilization agent, the permeabilization agent can be removed from contact with sample (e.g., by opening sample holder) before complete permeabilization of sample. For example, in some embodiments, only a portion of sample is permeabilized, and only a portion of the analytes or proxies thereof in the sample may be captured by the feature array. In some instances, the reduced amount of analytes or proxies thereof captured and available for detection can be offset by the reduction in lateral diffusion that results from incomplete permeabilization of sample. In general, the spatial resolution of the assay is determined by the extent of analytes or proxies thereof diffusion in the transverse direction (i.e., orthogonal to the normal direction to the surface of sample). The larger the distance between the sample on the first substrate and the feature array on the second substrate, the greater the extent of diffusion in the transverse direction, and the concomitant loss of resolution. Analytes or proxies thereof liberated from a portion of the sample closest to the feature array have a shorter diffusion path, and therefore do not diffuse as far laterally as analytes or proxies thereof from portions of the sample farthest from the feature array. As a result, in some instances, incomplete permeabilization of the sample (by reducing the contact interval between the permeabilization agent and the sample) can be used to maintain adequate spatial resolution in the assay.

In some instances, the device is configured to control a temperature of the first and second substrates. In some embodiments, the temperature of the first and second members is lowered to a first temperature that is below room temperature (e.g., 25 degrees Celsius) (e.g., 20 degrees Celsius or lower, 15 degrees Celsius or lower, 10 degrees Celsius or lower, 5 degrees Celsius or lower, 4 degrees Celsius or lower, 3 degrees Celsius or lower, 2 degrees Celsius or lower, 1 degree Celsius or lower, 0 degrees Celsius or lower, −1 degrees Celsius or lower, −5 degrees Celsius or lower). In some embodiments, the device includes a temperature control system (e.g., heating and cooling conducting coils) to control the temperature of the sample holder. Alternatively, in other embodiments, the temperature of the sample holder is controlled externally (e.g., via refrigeration or a hotplate). In a first step, the second member, set to or at the first temperature, contacts the first substrate, and the first member, set to or at the first temperature, contacts the second substrate, thereby lowering the temperature of the first substrate and the second substrate to a second temperature. In some embodiments, the second temperature is equivalent to the first temperature. In some embodiments, the first temperature is lower than room temperature (e.g., 25 degrees Celsius). In some embodiments, the second temperature ranges from about −10 degrees Celsius to about 4 degrees Celsius. In some embodiments, the second temperature is below room temperature (e.g., 25 degrees Celsius) (e.g., 20 degrees Celsius or lower, 15 degrees Celsius or lower, 10 degrees Celsius or lower, 5 degrees Celsius or lower, 4 degrees Celsius or lower, 3 degrees Celsius or lower, 2 degrees Celsius or lower, 1 degree Celsius or lower, 0 degrees Celsius or lower, −1 degrees Celsius or lower, −5 degrees Celsius or lower).

In an exemplary embodiment, the second substrate is contacted with the permeabilization reagent. In some embodiments, the permeabilization reagent is dried. In some embodiments, the permeabilization reagent is a gel or a liquid. Also in the exemplary embodiment, the biological sample is contacted with buffer. Both the first and second substrates are placed at lower temperature to slow down diffusion and permeabilization efficiency. Alternatively, in some embodiments, the sample can be contacted directly with a liquid permeabilization reagent without inducing an unwanted initiation of permeabilization due to the substrates being at the second temperature. In some embodiments, the low temperature slows down or prevents the initiation of permeabilization. In a second step, keeping the sample holder and substrates at a cold temperature (e.g., at the first or second temperatures) continues to slow down or prevent the permeabilization of the sample. In a third step, the sample holder (and consequently the first and second substrates) is heated up to initiate permeabilization. In some embodiments, the sample holder is heated up to a third temperature. In some embodiments, the third temperature is above room temperature (e.g., 25 degrees Celsius) (e.g., 30 degrees Celsius or higher, 35 degrees Celsius or higher, 40 degrees Celsius or higher, 50 degrees Celsius or higher, 60 degrees Celsius or higher). In some embodiments, analytes or proxies thereof that are released from the permeabilized tissue of the sample diffuse to the surface of the second substrate and are captured by released capture probes from the second substrate. In a fourth step, the first substrate and the second substrate are separated (e.g., pulled apart) and temperature control is stopped.

In some embodiments, where either the first substrate or substrate second (or both) includes wells, a permeabilization solution can be introduced into some or all of the wells, and then the sample and the feature array can be contacted by closing the sample holder to permeabilize the sample. In certain embodiments, a permeabilization solution can be soaked into a hydrogel film that is applied directly to the sample, and/or soaked into features (e.g., beads) of the array. When the first and second substrates are aligned in the sandwich configuration, the permeabilization solution promotes migration of analytes or proxies thereof from the sample to the array.

In certain embodiments, different permeabilization agents or different concentrations of permeabilization agents can be infused into array features (e.g., beads) or into a hydrogel layer as described above. By locally varying the nature of the permeabilization reagent(s), the process of analytes or proxies thereof capture from the sample can be spatially adjusted.

In some instances, migration of the analytes, e.g., target nucleic acids or proxies thereof, from the biological sample to the first and second region of capture probes arrayed on the second substrate is passive (e.g., via diffusion). Alternatively, in certain embodiments, migration of the analytes, e.g., target nucleic acids or proxies thereof, from the biological sample to the first and second region of capture probes arrayed on the second substrate is performed actively (e.g., electrophoretic, by applying an electric field to promote migration). In some instances, first and second substrates can include a conductive epoxy. Electrical wires from a power supply can connect to the conductive epoxy, thereby allowing a user to apply a current and generate an electric field between the first and second substrates. In some embodiments, electrophoretic migration results in higher capture efficiency of analytes or proxies thereof and better spatial fidelity of captured analytes or proxies thereof than random diffusion onto matched substrates without the application of an electric field (e.g., via manual alignment of the two substrates). Exemplary methods of electrophoretic migration are described in WO 2020/176788, including at FIGS. 13-15, 24A-24B, and 25A-25C, which is hereby incorporated by reference in its entirety.

Spatial analysis workflows can include a sandwiching process described herein. In some embodiments, the workflow includes provision of the first substrate comprising the biological sample. In some embodiments, the workflow includes, mounting the biological sample onto the first substrate. In some embodiments wherein the biological sample is a tissue sample, the workflow includes sectioning of the tissue sample (e.g., cryostat sectioning). In some embodiments, the workflow includes a fixation step. In some instances, the fixation step can include fixation with methanol. In some instances, the fixation step includes formalin (e.g., 2% formalin).

In some embodiments, the biological sample on the first substrate is stained using any of the methods described herein. In some instances, the biological sample is imaged, capturing the stain pattern created during the stain step. In some instances, the biological sample then is destained prior to the sandwiching process.

In some instances, the methods include imaging the biological sample. In some instances, imaging occurs prior to sandwich assembly. In some instances, imaging occurs while the sandwich configuration is assembled. In some instances, imaging occurs during permeabilization of the biological sample. In some instances, images are captured using high resolution techniques (e.g., having 300 dots per square inch (dpi) or greater). For example, images can be captured using brightfield imaging (e.g., in the setting of hematoxylin or H&E stain) or using fluorescence microscopy to detect adhered labels. In some instances, high resolution images are captured temporally using e.g., confocal microscopy. In some instances, a low resolution image is captured. A low resolution image (e.g., images that are about 72 dpi and normally have an RGB color setting) can be captured at any point of the workflow, including but not limited to staining, destaining, permeabilization, sandwich assembly, and migration of the analytes or proxies thereof. In some instances, a low resolution image is taken during permeabilization of the biological sample.

In some instances, the biological samples can be destained. In some instances, destaining occurs prior to permeabilization of the biological sample. By way of example only, H&E staining can be destained by washing the sample in HCl. In some instances, the hematoxylin of the H&E stain is destained by washing the sample in HCl. In some embodiments, destaining can include 1, 2, 3, or more washes in HCl. In some embodiments, destaining can include adding HCl to a downstream solution (e.g., permeabilization solution).

Between any of the methods disclosed herein, the methods can include a wash step (e.g., with SSC (e.g., 0.1×SSC)). Wash steps can be performed once or multiple times (e.g., 1×, 2×, 3×, between steps disclosed herein). In some instances, wash steps are performed for about 10 seconds, about 15 seconds, about 20 seconds, about 30 seconds, or about a minute. In some instances, three washes occur for 20 seconds each. In some instances, the wash step occurs before staining the sample, after destaining the sample, before permeabilization the sample, after permeabilization the sample, or any combination thereof.

In some instances, after the sandwiching process the first substrate and the second substrate are separated (e.g., such that they are no longer aligned in a sandwich configuration, also referred to herein as opening the sandwich). In some embodiments, subsequent analysis (e.g., cDNA synthesis, library preparation, and sequences) can be performed on the captured analytes or proxies thereof after the first substrate and the second substrate are separated. In some embodiments, the process of transferring the analytes, e.g., target nucleic acids or proxies thereof, from the biological sample to the first and second region of capture probes arrayed on the second substrate is referred to interchangeably herein as a “sandwich process,” “sandwiching process,” or “sandwiching”. The sandwich process is further described in PCT Patent Application Publication No. WO 2020/123320, PCT Publ. No. WO 2021/252747, and PCT Publ. No. WO 2022/061152, which are incorporated by reference in their entireties.

### D. Library Preparation

In some embodiments, the analytes (e.g., target nucleic acids) or proxies thereof (e.g., ligation products, extension products, analyte binding moiety barcodes of analyte capture agents), and/or amplicons of such products, can be prepared for downstream applications, such as generation of a sequencing library and next-generation sequencing. Generating sequencing libraries are known in the art. For example, the analyte, proxies of analytes and/or their complements thereof can be purified and collected for downstream amplification steps. The products can be amplified using PCR, where primer binding sites flank the spatial barcode and analyte, or a complement thereof, generating a library associated with a particular spatial barcode. In some embodiments, the library preparation can be quantitated and/or quality controlled to verify the success of the library preparation steps. The library amplicons are sequenced and analyzed to decode spatial information of the analyte or proxy thereof (e.g., a ligation product, an analyte capture agent).

Alternatively, or additionally, the amplicons can then be enzymatically fragmented and/or size-selected in order to provide for desired amplicon size. In some embodiments, when utilizing an Illumina® library preparation methodology, for example, P5 and P7, sequences can be added to the amplicons thereby allowing for capture of the library preparation on a sequencing flow cell (e.g., on Illumina sequencing instruments). Additionally, i7 and i5 can index sequences be added as sample indexes if multiple libraries are to be pooled and sequenced together. Further, Read 1 and Read 2 sequences can be added to the library preparation for sequencing purposes. The aforementioned sequences can be added to a library preparation sample, for example, via End Repair, A-tailing, Adaptor Ligation, and/or PCR. The cDNA fragments can then be sequenced using, for example, paired-end sequencing using TruSeq Read 1 and TruSeq Read 2 as sequencing primer sites, although other methods are known in the art.

Library preparation, in the present disclosure, is expected to be successful when amplifying extended capture probes from the first region of the array as the capture probes from this region of the array comprise functional sequences such as sequencing primer binding sequences and/or amplification domains. However, the capture probes from the second region lack these sequencing primer binding sequences and/or amplification domains, as such, a library from an extended capture probes from the second region is not expected.

### E. Kits

The present disclosure also features kits including substrates which include arrays including a first region of capture probes and a second region of capture probes. As described above, the second region of capture probes can surround or form a perimeter around the first region of capture probes.

Thus provided herein are kits including a spatial array, the spatial array including (a) a spatial array including a first plurality of capture probes, wherein each of the first plurality of capture probes includes a (i) a spatial barcode, (ii) a first capture domain, and (iii) one or more functional domains; and (b) a second plurality of capture probes, wherein each of the second plurality of capture probes includes a second capture domain; wherein the second plurality of capture probes are configured to surround the first plurality of capture probes on the array. In some embodiments, each of the second plurality of capture probes does not include a spatial barcode or a functional domain (e.g., a sequencing specific site or a primer binding site).

In some embodiments, the kit further includes a polymerase. The polymerase can be a reverse transcriptase, a ligase, and/or a DNA polymerase. In some embodiments, each of the first plurality of capture probes further includes a unique molecular identifier (UMI), a cleavage domain, or combinations thereof. In some embodiments, the kit includes one or more permeabilization reagents. In some embodiments, the one more permeabilization reagents include one or more of a protease, a lipase, a DNase, an RNase, a detergent, and combinations thereof. In some embodiments, the protease comprises pepsin or proteinase K. In some embodiments, the kit further includes instructions for performing any of the methods of disclosed herein.

### F. Compositions

The present disclosure also features compositions including arrays including a first region of capture probes and a second region of capture probes. As described above, the second region of capture probes can surround or form a perimeter around the first region of capture probes.

This provided herein are compositions including a spatial array including: a) a first plurality of capture probes, where each of the first plurality of capture probes comprises (i) a spatial barcode, (ii) a first capture domain, and (iii) one or more functional domains (e.g., any of the functional domains described herein); (b) a second plurality of capture probes, where each of the second plurality of capture probes comprises a second capture domain, and where the second plurality of capture probes comprise the perimeter of the array. In some embodiments of the spatial array, the perimeter includes the peripheral edge of a plurality of patterned features on the spatial array.

In some embodiments of the spatial array, the spatial array includes (c) a biological sample including a plurality of target nucleic acids on the array; and (d) a first probe and a second probe hybridized to the target nucleic acid and ligated together, where the first probe and the second probe each comprise a sequence that is substantially complementary to adjacent sequences of the target nucleic acid, wherein the first probe and/or the second probe contain one or more unique barcode sequences selected from a plurality of barcode sequences, and wherein one of the first probe or the second probe comprises a capture probe capture domain (e.g., a sequence complementary to a capture domain (i.e., a first capture domain, a second capture domain).

In some embodiments, each of the first plurality of capture probes includes a unique molecular identified (UMI). In some embodiments, each of the second plurality of capture probes does not include a spatial barcode or functional domain (e.g., a sequencing specific site or a primer binding site of the capture probes in the first region). In some embodiments, each of the first plurality of capture probes and each of the second plurality of capture probes includes a poly(T) capture domain sequence. In some embodiments, each of the first plurality of capture probes includes one or more functional domains, a cleavage domain, or a combination thereof.

### G. Embodiments

Embodiment 1 is method for reducing mislocalization of target nucleic acids from a biological sample, the method comprising: (a) providing an array, wherein the array comprises a first region of capture probes and a second region of capture probes, wherein capture probes of the first region of capture probes comprise: (i) a spatial barcode, (ii) a capture domain, and (iii) one or more functional domains, and capture probes of the second region of capture probes comprise a second capture domain; and (b) hybridizing target nucleic acids from the biological sample to the capture domains of capture probes of the first region and the capture probes of the second region, such that hybridization of the target nucleic acids to the capture probes of the second region of capture probes reduces mislocalization of target nucleic acids.

Embodiment 2 is the method of embodiment 1, further comprising migrating the target nucleic acids from the biological sample to the array.

Embodiment 3 is the method of embodiment 1 or 2, wherein the biological sample is disposed on the array.

Embodiment 4 is the method of embodiment 1 or 2, wherein the biological sample is disposed on a substrate.

Embodiment 5 is the method of embodiment 4, wherein the method further comprises aligning the substrate comprising the biological sample with the array, such that at least a portion of the biological sample is aligned with at least a portion of the first region on the array.

Embodiment 6 is the method of any one of embodiments 1-5, wherein the one or more functional domains is a primer binding site or a sequencing specific site.

Embodiment 7 is the method of any one of embodiments 1-6, wherein capture probes of the second region do not have one or more of a primer binding site or a sequencing specific site.

Embodiment 8 is the method of any one of embodiments 1-7, wherein capture probes of the first region further comprise a unique molecular identifier and/or a cleavage domain.

Embodiment 9 is the method of any one of embodiments 1-8, wherein the second region of capture probes surrounds the first region of capture probes.

Embodiment 10 is the method of any one of embodiments 1-8, wherein the capture probes of the first region and the capture probes of the second region are located at features on the array.

Embodiment 11 is the method of any one of embodiments 1-10, wherein the capture domains of the capture probes are the same in the first region and the second region and comprise a poly(T) sequence.

Embodiment 12 is the method of any one of embodiments 1-10, wherein the capture domains of the capture probes are the same in the first region and the second region and comprise a non-homopolymeric sequence.

Embodiment 13 is the method of any one of embodiments 1-12, wherein the target nucleic acids are mRNA.

Embodiment 14 the method of any of embodiments 1-13, wherein the target nucleic acids are ligation products that represents mRNA target nucleic acids.

Embodiment 15 is the method of embodiment 14, wherein the ligation products are generated by ligating first probes that hybridize to the target nucleic acids and second probes that hybridize adjacently to the first probes on the target nucleic acids.

Embodiment 16 is the method of embodiment 15, wherein either the first probes or the second probes comprise a capture domain that is complementary to the capture domain of the capture probes on the array, and either the first probes or the second probes comprise one or more of a primer binding site or a sequencing specific site.

Embodiment 17 is the method of any one of embodiments 15-16 wherein the ligating is performed by a ligase selected from a PBCV-1 ligase, a Chlorella DNA ligase, a single stranded DNA ligase, or a T4 DNA ligase.

Embodiment 18 is the method of embodiment 17, wherein the ligase is T4 DNA ligase or a Chlorella DNA ligase.

Embodiment 19 is the method of any one of embodiments 1-18, further comprising determining (i) the sequence of the spatial barcode or a complement thereof, and (ii) all or a portion of the sequence of the target nucleic acids and using the determined sequences of (i) and (ii) to determine the location of the target nucleic acids in the biological sample.

Embodiment 20 is the method of embodiment 19, wherein the determining comprises sequencing.

Embodiment 21 is the method of embodiment 20, wherein the sequencing comprises high-throughput sequencing.

Embodiment 22 is a method of reducing sequencing output from mislocalized target nucleic acids from a biological sample, the method comprising: (a) providing an array, wherein the array comprises a first region of capture probes and a second region of capture probes, wherein capture probes of the first region comprise: (i) a spatial barcode, (ii) a capture domain, and (iii) one or more functional domains, and capture probes of the second region comprise a capture domain; (b) contacting target nucleic acids from the biological sample with the plurality of capture probes on the array wherein the capture probes of the first and second regions hybridize to the target nucleic acids in the first and second regions proximal to their location in the biological sample; (c) extending the capture probes hybridized to the target nucleic acids; and (d) determining the sequence of the (i) spatial barcode or a complement thereof and (ii) a portion of the target nucleic acid or a complement thereof from the capture probes in the first region, thereby reducing sequencing output for mislocalized target nucleic acids from the biological sample.

Embodiment 23 is the method of embodiment 22, wherein the capture probes of the second region are not sequenced and wherein the second region is located around a perimeter of the array and surrounds the first region.

Embodiment 24 is the method of embodiment 22 or 23, wherein one or more portions of the biological sample is located over the second region of the array.

Embodiment 25 is the method of any one of embodiments 22-24, further comprising migrating the target nucleic acids from the biological sample to the array.

Embodiment 26 is the method of embodiment 24, wherein the method further comprises aligning a substrate comprising the biological sample with the array, such that at least a portion of the biological sample is aligned with at least a portion of the first region of the array.

Embodiment 27 is the method of any one of embodiments 22-26, wherein the one or more functional domains is a primer binding site or a sequencing specific site.

Embodiment 28 is the method of any one of embodiments 22-27, wherein capture probes of the second region do not have a primer binding site or a sequencing specific site.

Embodiment 29 is the method of any one of embodiments 22-28, wherein capture probes of the first region further comprise one or more of a unique molecular identifier (UMI) and a cleavage domain.

Embodiment 30 the method of any one of embodiments 22-29, wherein the capture probes of the first region and the capture probes of the second region are affixed to features on the array.

Embodiment 31 is the method of embodiment 30, wherein the capture probes of the first region are affixed to features in an interior of the array and capture probes of the second region are affixed to features on the perimeter of the array.

Embodiment 32 is the method of any one of embodiments 22-31, wherein the capture domains of the capture probes are the same in the first region and the second region and comprise a poly(T) sequence.

Embodiment 33 the method of any one of embodiments 22-31, wherein the capture domains of the capture probes are the same in the first region and the second region and comprise a non-homopolymeric sequence.

Embodiment 34 the method of any one of embodiments 22-33, wherein the target nucleic acid is mRNA.

Embodiment 35 is the method of any one of embodiments 22-33, wherein the target nucleic acid is a ligation product that represents a mRNA target nucleic acid.

Embodiment 36 is the method of embodiment 35, wherein the ligation product is generated by ligating a first probe that hybridizes to the target nucleic acid sequence and a second probe that hybridizes adjacently to the first probe on the target nucleic acid sequence.

Embodiment 37 is the method of embodiment 36, wherein either the first probe or second probe comprises a capture probe binding domain that is complementary to the capture domain of the capture probes on the array.

Embodiment 38 is the method of embodiment 37, wherein either the first probe or second probe further comprises one or more of a primer binding site or a sequencing specific site.

Embodiment 39 is the method of any one of embodiments 36-38, wherein the ligating is performed by a ligase selected from a PBCV-1 ligase, a Chlorella DNA ligase, a single stranded DNA ligase, or a T4 DNA ligase.

Embodiment 40 is the method of embodiment 39, wherein the ligase is T4 DNA ligase or a Chlorella DNA ligase.

Embodiment 41 is the method of any one of embodiments 1-40, wherein the biological sample is a tissue sample.

Embodiment 42 is the method of embodiment 41, wherein the tissue sample is a tissue section.

Embodiment 43 is the method of embodiment 41, wherein the tissue sample is a fixed tissue sample.

Embodiment 44 is the method of embodiment 43, wherein the fixed tissue sample is methanol-fixed, acetone-fixed, PFA-fixed, or is formalin-fixed paraffin-embedded (FFPE).

Embodiment 45 is the method of embodiment 44, wherein the FFPE tissue is deparaffinized and decrosslinked prior to step (a).

Embodiment 46 is the method of embodiment 41, wherein the tissue sample is a fresh frozen tissue sample.

Embodiment 47 is the method of embodiment 41, wherein the tissue sample is fixed and stained prior to step (a) using immunofluorescence, immunohistochemistry, or hematoxylin and eosin.

Embodiment 48 is the method of any one of embodiments 1-47, further comprising imaging the biological sample.

Embodiment 49 is the method of any one of embodiments 1-48, further comprising permeabilizing the biological sample.

Embodiment 50 is the method of embodiment 49, wherein the permeabilizing comprises use of a protease.

Embodiment 51 is the method of embodiment 50, wherein the protease comprises pepsin.

Embodiment 52 is the method of embodiment 50, wherein the protease comprises proteinase K.

Embodiment 53 is a spatial array comprising: (a) a first plurality of capture probes, wherein each of the first plurality of capture probes comprises (i) a spatial barcode, (ii) a capture domain, and (iii) one or more functional domains; and (b) a second plurality of capture probes, wherein each of the second plurality of capture probes comprises a capture domain, and wherein the second plurality of capture probes are positionally located around a perimeter of the array.

Embodiment 54 is the spatial array of embodiment 53, wherein the perimeter includes a peripheral edge of a plurality of patterned features on the spatial array.

Embodiment 55 is the spatial array of embodiment 53 or 54, further comprising: (c) a biological sample comprising a plurality of target nucleic acids on the array; and (d) first probe and a second probe hybridized to the target nucleic acid and ligated together, wherein the first probe and the second probe each comprise a sequence that is substantially complementary to adjacent sequences of the target nucleic acid, wherein the first probe and/or the second probe contain one or more functional sequences, and wherein one of the first probe or the second probe comprises a capture probe capture domain.

Embodiment 56 is the spatial array of any one of embodiments 53-55, wherein each of the first plurality of capture probes further comprises a unique molecular identified (UMI).

Embodiment 57 is the spatial array of any one of embodiments 53-56, wherein the second plurality of capture probes does not comprise a spatial barcode or a functional domain.

Embodiment 58 is the spatial array of any one of embodiments 53-57, wherein each of the first plurality of capture probes and each of the second plurality of capture probes comprises a poly(T) sequence.

Embodiment 59 is the spatial array of any one of embodiments 53-58, wherein each of the first plurality of capture probes comprises one or more functional domains, a cleavage domain, and combinations thereof.

Embodiment 60 is the spatial array of any one of embodiments 53-59, wherein the first plurality of capture probes and the second plurality of capture probes are affixed to the plurality of patterned features on the array.

Embodiment 61 is a kit comprising: (a) a spatial array comprising a first plurality of capture probes, wherein each of the first plurality of capture probes comprises a (i) a spatial barcode, (ii) a capture domain, and (iii) one or more functional domains; and (b) a second plurality of capture probes, wherein each of the second plurality of capture probes comprises a capture domain; wherein the second plurality of capture probes are configured to surround the first plurality of capture probes on the array.

Embodiment 62 is the kit of embodiment 61, wherein each of the second plurality of capture probes does not comprise a spatial barcode or a functional domain.

Embodiment 63 is the kit of embodiment 61 or 62, further comprising a polymerase.

Embodiment 64 is the kit of embodiment 63, wherein the polymerase comprises one of a reverse transcriptase, a DNA polymerase and a ligase.

Embodiment 65 is the kit of any one of embodiments 61-64, wherein each of the first plurality of capture probes further comprises a unique molecular identifier (UMI), a cleavage domain, or combinations thereof.

Embodiment 66 is the kit of any one of embodiments 61-65, wherein the kit further comprises one or more permeabilization reagents.

Embodiment 67 is the kit of embodiment 66, wherein the one more permeabilization reagents comprising pepsin or proteinase K.

Embodiment 68 is the kit of embodiment 61, further comprising a RNase.

Embodiment 69 is the kit of embodiment 68, wherein the RNase is RNase H.

Embodiment 70 is the kit of any one of embodiments 61-68, further comprising instructions for performing any of the methods of embodiments 1-52.

What is claimed is:

1. A method for reducing mislocalization of target nucleic acids from a biological sample, the method comprising:
(a) providing an array, wherein the array comprises a first region of capture probes and a second region of capture probes, wherein capture probes of the first region of capture probes comprise: (i) a spatial barcode, (ii) a first capture domain, and (iii) one or more functional domains comprising a primer binding site or a sequencing specific site, and capture probes of the second region of capture probes comprise a second capture domain; and
(b) hybridizing target nucleic acids from the biological sample to the first capture domain of capture probes of the first region and a second capture domain of capture probes of the second region, wherein the capture probes of the second region do not comprise one or more of a primer binding site or a sequencing specific site, such that hybridization of the target nucleic acids to the capture probes of the second region of capture probes reduces mislocalization of target nucleic acids.

2. The method of claim 1, further comprising migrating the target nucleic acids from the biological sample to the array.

3. The method of claim 1, wherein the biological sample is disposed on the array.

4. The method of claim 1, wherein the biological sample is disposed on a first substrate.

5. The method of claim 4, wherein the method further comprises aligning the first substrate comprising the biological sample with a second substrate comprising the array, such that at least a portion of the biological sample is aligned with at least a portion of the first region on the array.

6. The method of claim 1, wherein capture probes of the first region further comprise a unique molecular identifier and/or a cleavage domain.

7. The method of claim 1, wherein the second region of capture probes surrounds the first region of capture probes, optionally wherein the capture probes of the first region are affixed to features in an interior of the array and capture probes of the second region are affixed to features on the perimeter of the array.

8. The method of claim 7, wherein the perimeter of the array includes a peripheral edge of a plurality of patterned features on the array.

9. The method of claim 1, wherein the first capture domain is the same as the second capture domain, optionally wherein the first capture domain and the second capture domain independently comprise a poly(T) sequence.

10. The method of claim 1, wherein the target nucleic acids comprise mRNA.

11. The method of claim 1, further comprising extending the capture probes of the first region using the target nucleic acids as templates, thereby generating extension products and, optionally, generating complementary strands to the extension products.

12. The method of claim 1, wherein the target nucleic acids are ligation products that represents mRNA target nucleic acids.

13. The method of claim 12, wherein the ligation products are generated by ligating first probes that hybridize to the target nucleic acids and second probes that hybridize adjacently to the first probes on the target nucleic acids.

14. The method of claim 13, wherein either the first probes or the second probes comprise a capture domain that is complementary to the first capture domain and/or the second capture domain on the array, and either the first probes or the second probes comprise one or more of a primer binding site or a sequencing specific site.

15. The method of claim 1, further comprising determining: (i) the sequence of the spatial barcode or a complement thereof, and (ii) all or a portion of the sequence of the target nucleic acids, or a complement thereof, or all or a portion of the sequence of the ligation products, or a complement thereof, and using the determined sequences of (i) and (ii) to determine the locations of the target nucleic acids in the biological sample.

16. The method of claim 1, further comprising generating a nucleic acid library for sequencing and sequencing the nucleic acid library with a sequencing primer that hybridizes to the sequencing specific site of capture probes in the first region.

17. A method of reducing sequencing output from mislocalized target nucleic acids from a biological sample, the method comprising:
(a) providing an array, wherein the array comprises a first region of capture probes and a second region of capture probes, wherein capture probes of the first region comprise: (i) a spatial barcode, (ii) a first capture domain, and (iii) one or more functional domains, and capture probes of the second region comprise a second capture domain and do not comprise one or more functional domains;
(b) contacting target nucleic acids from the biological sample with the plurality of capture probes on the array, wherein the capture probes of the first and second regions hybridize to the target nucleic acids in the first and second regions proximal to their location in the biological sample;
(c) extending the capture probes hybridized to the target nucleic acids, thereby generating extension products; and
(d) determining the sequence of the (i) spatial barcode or a complement thereof and (ii) a portion of the target nucleic acid or a complement thereof from the extension products from capture probes in the first region, thereby reducing sequencing output for mislocalized target nucleic acids from the biological sample.

18. The method of claim 17, wherein the one or more functional domains is a primer binding site or a sequencing specific site.

19. A spatial array comprising:
(a) a first plurality of capture probes, wherein each of the first plurality of capture probes comprises: (i) a spatial barcode, (ii) a first capture domain, and (iii) one or more functional domains; and
(b) a second plurality of capture probes, wherein each of the second plurality of capture probes comprises a second capture domain does not comprise one or more functional domains, and wherein the second plurality of capture probes are positionally located around a perimeter of the spatial array.

20. The spatial array of claim 19, wherein the one or more functional domains is a primer binding site or a sequencing specific site.
